(19) United States
(12) Patent Application Publication
(10) Pub. No.: US 2026/0069670 A1
(43) Pub. Date: Mar. 12, 2026

(54) ALPHA-HEMOLYSIN COMPOSITIONS AND METHODS FOR IMMUNIZATION AGAINST STAPHYLOCOCCUS AUREUS

(71) Applicant: Washington University, St. Louis, MO (US)

(72) Inventors: Juliane Bubeck-Wardenburg, St. Louis, MO (US); Kelly Tomaszewski, St. Louis, MO (US); Reuben Olaniyi, St. Louis, MO (US)

(21) Appl. No.: 19/242,516

(22) Filed: Jun. 18, 2025

Related U.S. Application Data
(63) Continuation of application No. PCT/US2024/024985, filed on Apr. 17, 2024.
(60) Provisional application No. 63/459,785, filed on Apr. 17, 2023.

(51) Int. Cl. A61K 39/085; A61K 39/00; A61P 37/04; C07K 14/31

(52) U.S. Cl. CPC A61K 39/085; A61P 37/04; C07K 14/31; A61K 2039/545; A61K 2039/55561

(57) ABSTRACT

Provided herein are compositions and methods for the treatment of Staphylococcus aureus.

### ACKNOWLEDGEMENT OF GOVERNMENT SUPPORT

This invention was made with government support under A1167137 awarded by the National Institutes of Health. The government has certain rights in the invention.

### FIELD

The present invention relates to compositions and methods for the treatment of Staphylococcus aureus.

### REFERENCE TO THE SEQUENCE LISTING

This application contains a Sequence Listing that has been submitted electronically as a computer readable XML format via Patent Center and is hereby incorporated by reference in its entirety pursuant to 37 C.F.R. § 1.52(e)(5). The XML file, created on Apr. 11, 2024, is named “047563-792748(020578).xml” and is 51.6 kilobytes in size.

### BACKGROUND

Staphylococcus aureus infection results in over 1 million deaths globally per year, afflicting individuals across the lifespan and disproportionately impacting those in low- and middle-income countries. The organism, S. aureus is both a human skin commensal and a leading cause of infection. Skin and soft tissue infection (SSTI) remain the most common form of S. aureus disease with an incidence of >100 cases per 100,000, costing >$4 billion/year in the U.S. SSTI's can lead to disseminated disease and have exacerbated the health burden of antibiotic-resistance.

While vaccine-based prevention of S. aureus infection is a public health necessity, prior efforts to develop an effective vaccine have failed. Each candidate vaccine to reach human clinical trials has been highly immunogenic, however the elicited immune response was not a known correlate of human protective immunity against S. aureus infection. In addition, recent studies have indicated that the pre-existing immune response to S. aureus is detrimental to vaccine efficacy. Novel approaches to vaccine development are needed, grounded in an understanding of human immunity to this pathogen and cognizant of the demand for a global solution.

### SUMMARY OF THE INVENTION

Disclosed herein is a modified α-hemolysin (Hla) polypeptide or a derivative or fragment thereof, comprising amino acid substitutions H35L, R66C and E70C (HlaHRE), relative to the amino acid sequence set forth in SEQ ID NO: 1. The modified Hla polypeptide may comprise the amino acid sequence set forth in SEQ ID NO: 11, or a sequence at least about 80% identical thereto, or a fragment thereof. The modified Hla polypeptide may comprise the amino acid sequence set forth in any one of SEQ ID NOS: 12-35, or a fragment thereof, or an amino acid sequence at least about 80% identical thereto.

Disclosed herein is a polynucleotide comprising a nucleic acid sequence encoding the modified Hla polypeptide. The modified Hla polypeptide may comprise the amino acid sequence as set forth in SEQ ID NO: 11, or a fragment thereof, or an amino acid sequence at least about 80% identical thereto. The polynucleotide sequence may be an isolated polynucleotide sequence, a plasmid, an expression vector, a cosmid, a viral vector. The polynucleotide sequence may be contained in a virus, or a virus like particle (VLP).

Also, disclosed is a host cell comprising the polynucleotide sequence. Non-limiting examples of host cells include Chinese hamster ovary (CHO) cell, a HEK 293 cell, a human cervical carcinoma cell (Hela), a canine kidney cell (MDCK), a human liver cell (HepG2), a baby hamster kidney cell (BHK), a monkey kidney cell (CV1), a Vero cell, a CEM cell, a 721.221 cell, a H9 cell, a Jurkat cell, a Raji cell, a W138 cell, a COS-7 cell, a 293 cell, a HepG2 cell, a 3T3 cell, and a RIN cell.

Disclosed herein are immunogenic compositions comprising the modified Hla polypeptide or a derivative or fragment thereof, or the polynucleotide sequence encoding the Hla polypeptide, and at least a pharmaceutically acceptable excipient. The immunogenic composition may further comprise a pharmaceutically acceptable adjuvant. Non-limiting examples of suitable adjuvants include alum, AddaSO3 (ASO3-like), MPLA and alum (ASO4-like), Fruend's, CpG-ODN1585, and any combination thereof. The immunogenic composition may further comprise at least one additional active agent.

Further disclosed herein is a method of inducing an immune response against a bacterial pathogen in a subject in need thereof, comprising administering to the subject the modified Hla polypeptide. Also disclosed are use of the modified Hla polypeptide in the manufacture of a medicament for inducing an immune response against the bacterial pathogen and the modified Hla polypeptide for inducing an immune response against the bacterial pathogen. The bacterial pathogen may be Staphylococcus aureus. Administering the modified Hla polypeptide to the subject may result in enhanced germinal center (GC) or T follicular helper (TFH) response or reduce the severity of skin and soft tissue infection, invasive S. aureus disease, sepsis, and carriage, as compared to administering an immunogenic composition comprising a modified Hla polypeptide with only a substitution H35L relative to the amino acid sequence set forth in SEQ ID NO: 1.

In an aspect, the subject may be a mammal. In an aspect, the subject may be a human. The subject may be of any age. In an aspect, the subject may be a child under 3 years of age, or under 2 years of age, or under 1 year of age, or less than 12 months, or 11 months, or 10 months, of 9 months, or 8 months, or 6 months, or 5 months, or 4 months, or 3 months, or 2 months, or 1 month, or less than 4 wks, or 3 wks, or 2 wks, or 1 wks in age. The subject may be a pregnant woman. The method may further comprise at least 1, at least 2, at least 3 or more additional administration of the immunogenic composition. The method may comprise administering at least one additional immunogenic antigen. Non-limiting examples of additional immunogenic antigens include Opp3a, DItD, HtsA, LtaS, IsdA, IsdB IsdC, SdrC, SdrD, SdrE, SdrF, SdrG, SdrH, SrtA, SpA, Sbi, FmtB, beta-hemolysin, fibronectin-binding protein A (FnbA), fibronectin-binding protein B (FnbB), coagulase, Fig, Map, Panton-Valentine leukocidin (Pvl), alpha-toxin and its variants, gamma toxin (hlg) and variants, Ica, immunodominant ABC transporter, Mg2+transporter, Ni-ABC transporter, RAP, autolysin, laminin receptors, IsaA/PisA, IsaB/PisB, SPOIIIE, SsaA, EbpS, Sas A, SasF, SasH, EFB (FIB), SBI, Npase, EBP, bone sialo binding protein II, aureolysin precursor (AUR)/Sepp1, CNA, and fragments thereof such as M55, TSST-1, mecA, poly-N-acetylglucosamine (PNAG/dPNAG) exopolysaccharide, GehD, EbhA, EbhB, SSP-1, SSP-2, HBP, vitronectin binding protein, HarA, EsxA, EsxB, Enterotoxin A, Enterotoxin B, Enterotoxin C1, and novel autolysin. The administration may be through an intravenous, intramuscular, sub-cutaneous, oral or intraperitoneal route.

### BRIEF DESCRIPTION OF THE DRAWINGS

The patent or application file contains at least one drawing executed in color. Copies of this patent or patent application with color drawing(s) will be provided by the Office by request and payment of the necessary fee. Aspects of the present disclosure are illustrated by way of example in which:

FIG. 1 provides a structural representation of the ADAM10 ectodomain (PDB 6BE6) showing structural orientation to the cellular membrane of each domain: metalloprotease (magenta), disintegrin (cyan) and cysteine-rich (green). Transmembrane and cytoplasmic tail domains are illustrated in gray.

FIG. 2A-2B provide an analysis of Hla-targeting vaccine candidate antigens. FIG. 2A provides an analysis of the serologic anti-Hla titer by age revealed a gradual increase in the half-maximal effective concentration (EC₅₀) over the first five years of life. 75^{th} and 25^{th} (black) and 50th (red) percentiles noted. *p<0.05, **p<0.01, ***p<0.001, ****p<0.0001, by one-way ANOVA and Tukey test for multiple comparisons. FIG. 2B provides an analysis of the functional Hla neutralizing activity of the sera in a rabbit red cell protection assay confirmed that infants and children under two years of age exhibit immaturity of the response relative to older children. 75^{th} and 25^{th} (black) and 50th (red) percentiles noted. *p<0.05, **p<0.01, ***p<0.001, ****p<0.0001, by one-way ANOVA and Tukey test for multiple comparisons.

FIG. 3A-3D provide an analysis of Hla-targeting candidate antigens. FIG. 3A Structural representation of monomeric Hla antigens (left, PDB 4YHD) and an isolated monomer from the heptameric toxin (right, PDB 7AHL) noting the location of variant residues in full-length vaccine antigens Hla_{H35L} (purple), Hla_{D45A/Y118F} (orange), Hla_{R66C/E70C} (blue). FIG. 3B provides a panel of peptide antigens: Hla₅₀ (SEQ ID NO: 36); Hla_{P1}: a synthetic antigen harboring 5 replicates of an IEDB-predicted T cell epitope within the first 50 amino acids of Hla (SEQ ID NO: 37); and Hla_{P2}: a synthetic antigen harboring three distinct predicted T cell antigenic peptides from Hla (SEQ ID NO: 38). FIG. 3C provides vaccine efficiency data for preventing abscesses for each antigen.

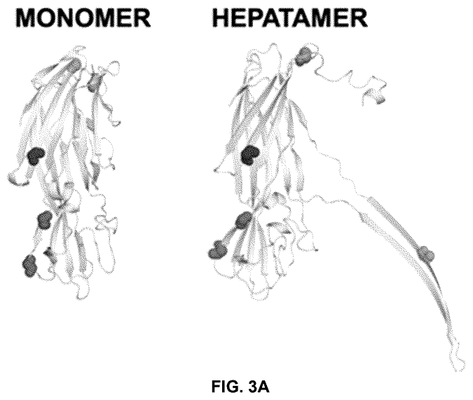

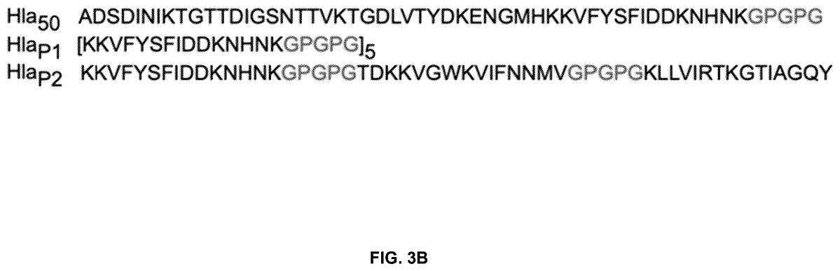

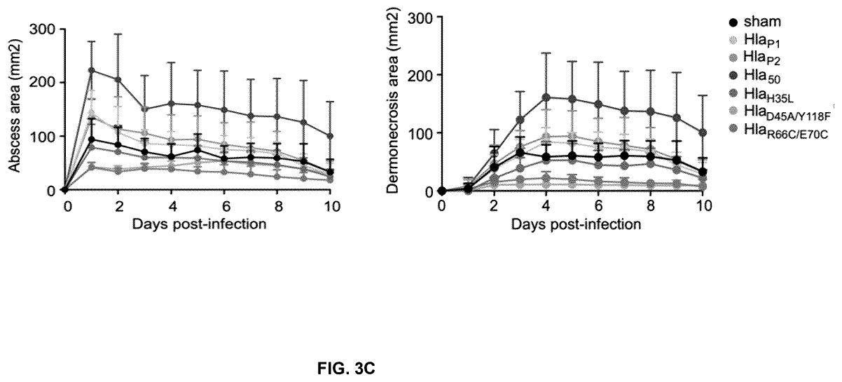

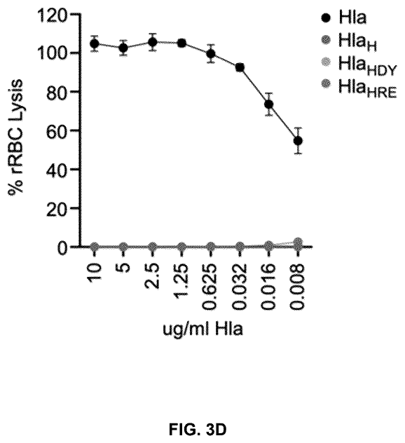

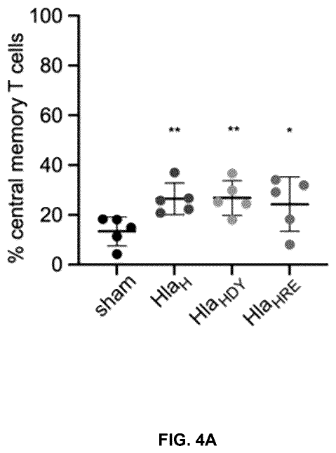

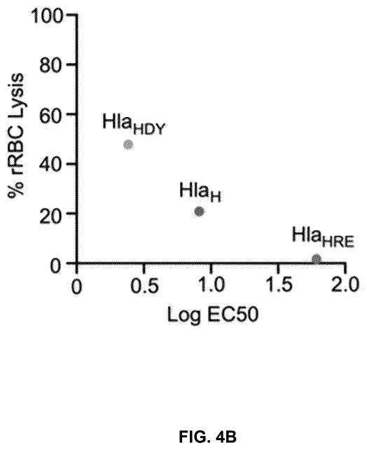

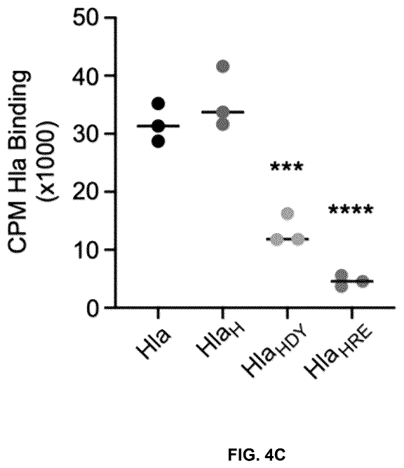

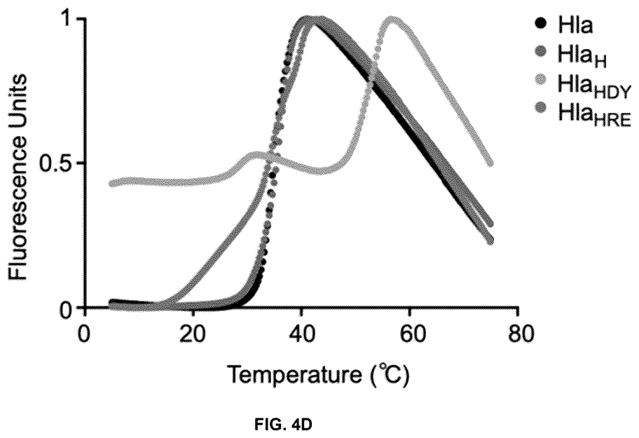

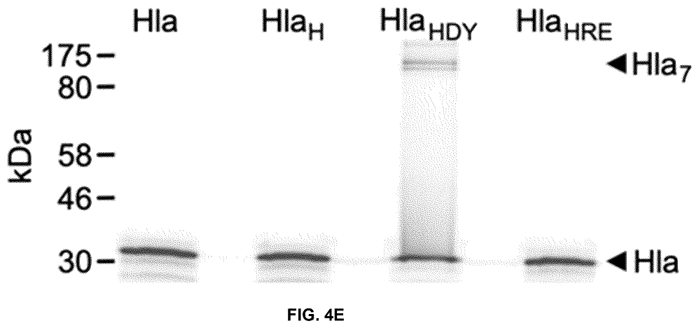

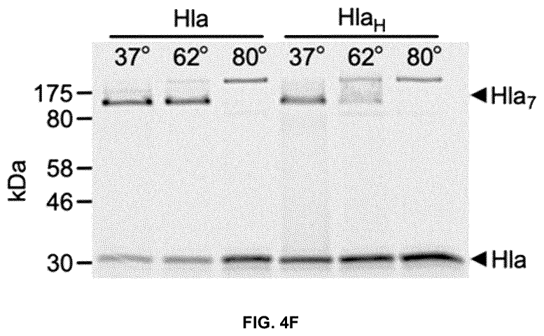

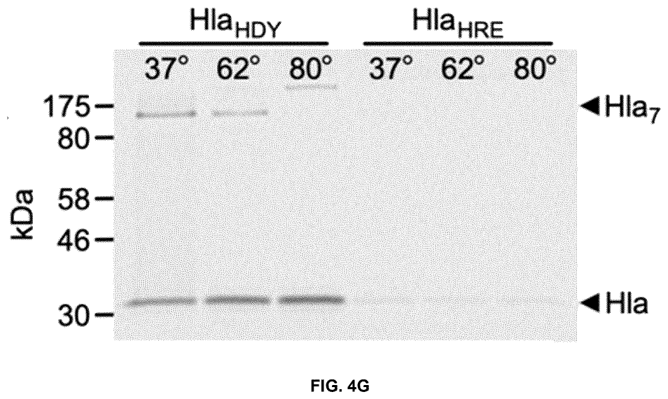

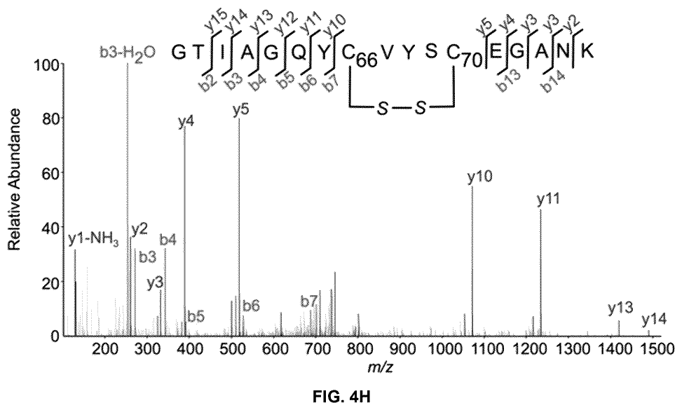

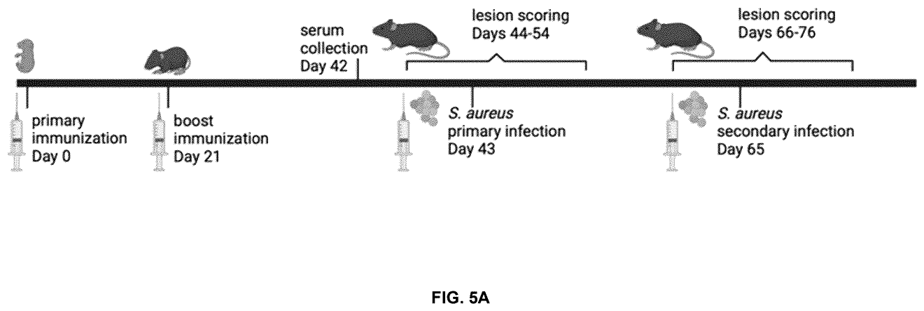

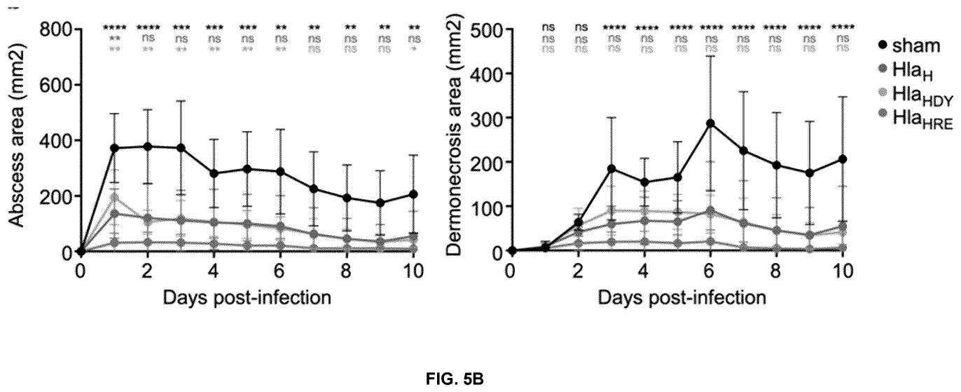

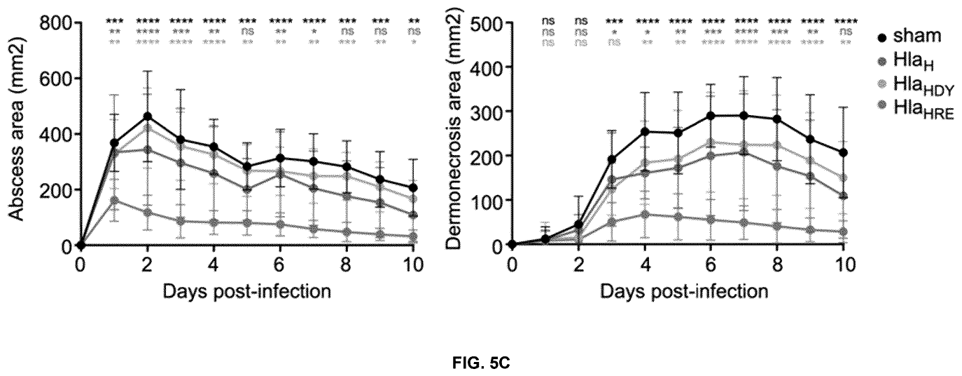

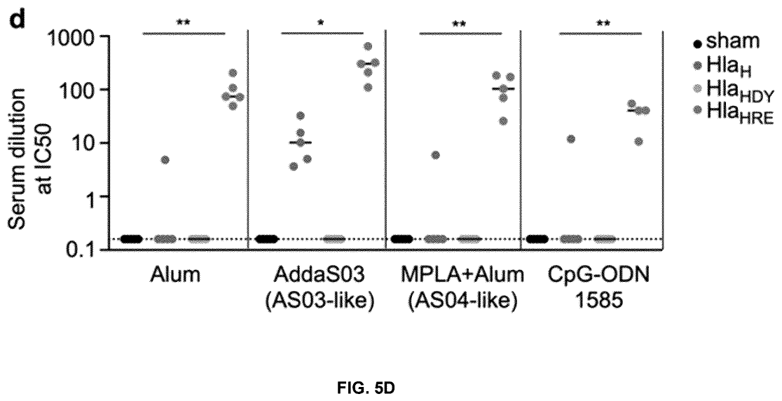

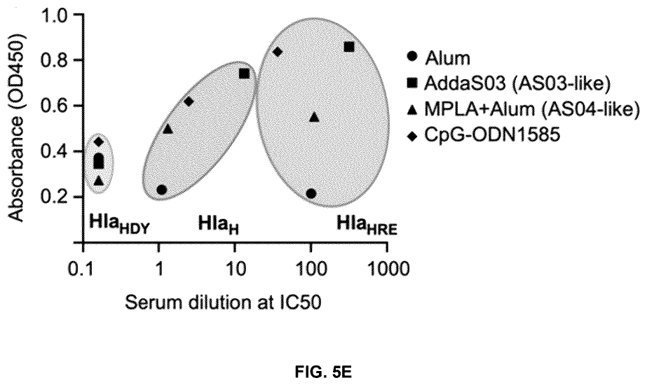

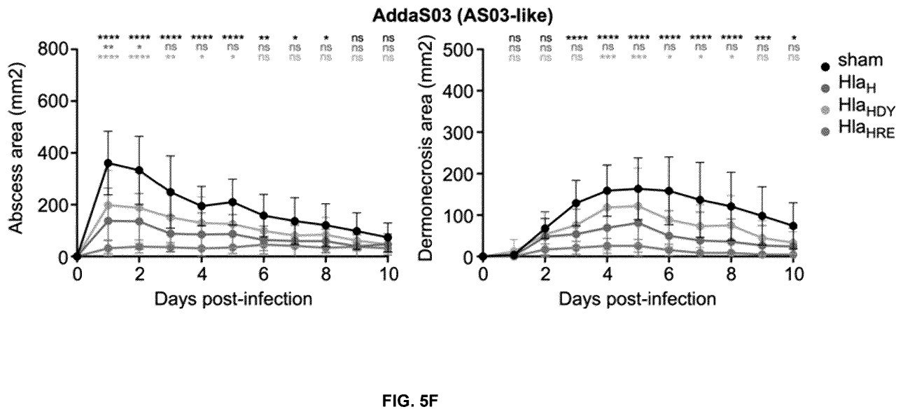

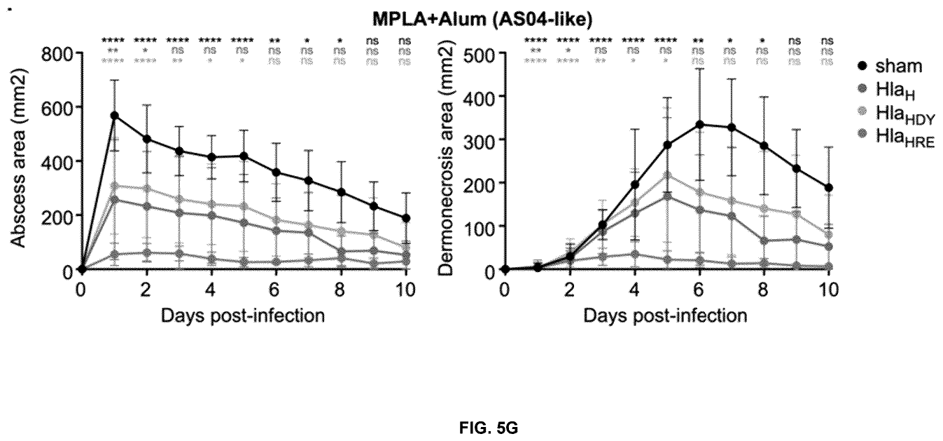

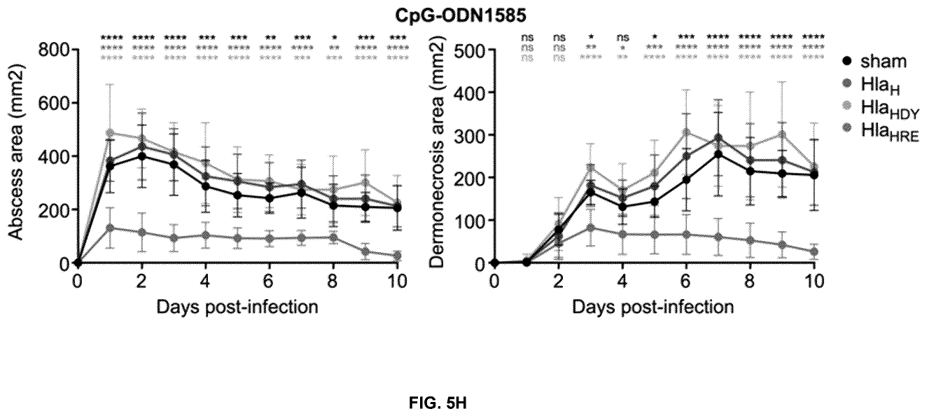

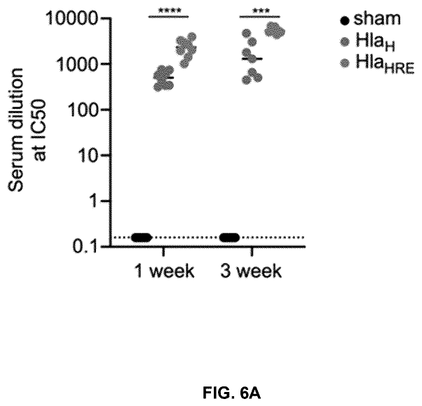

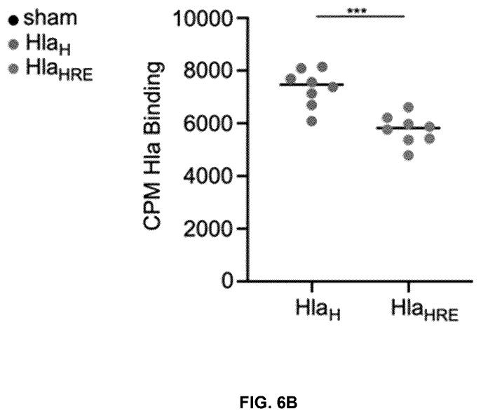

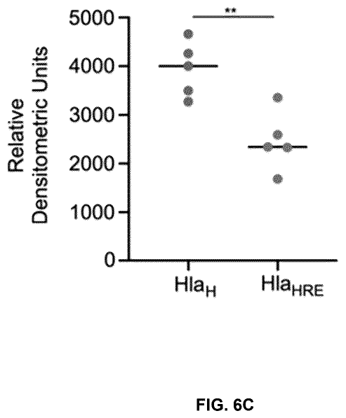

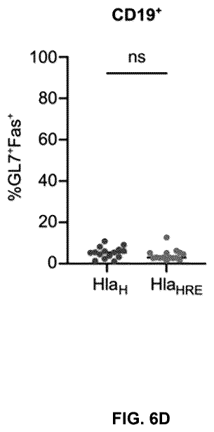

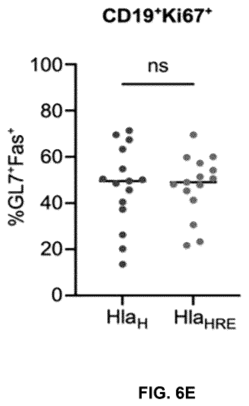

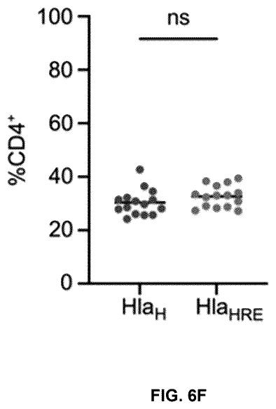

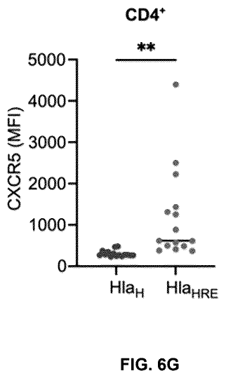

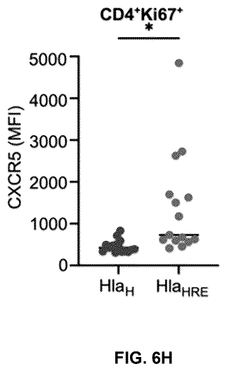

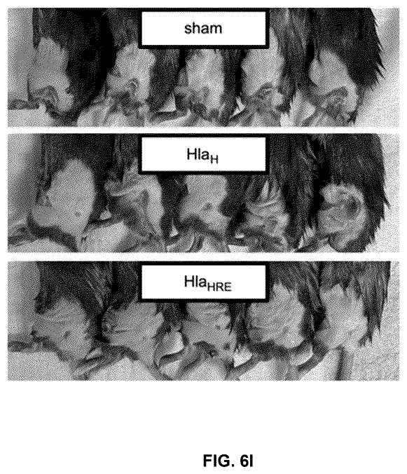

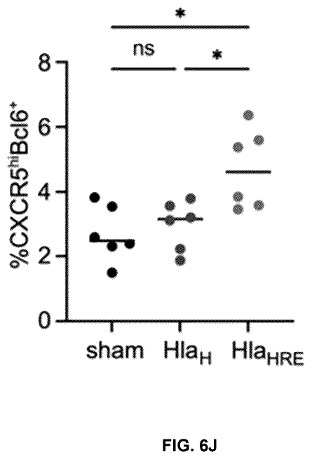

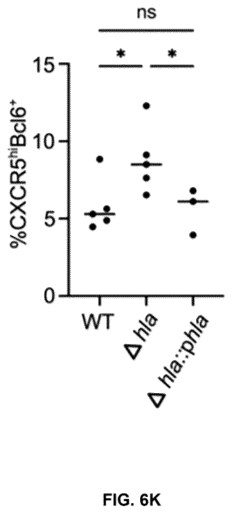

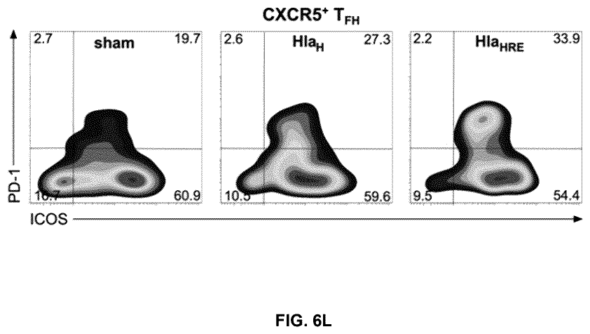

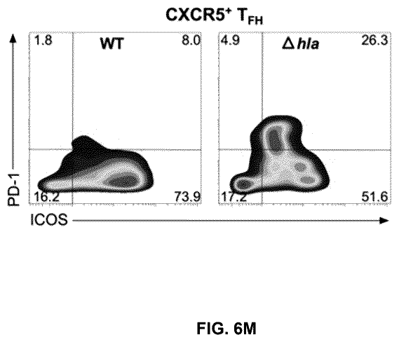

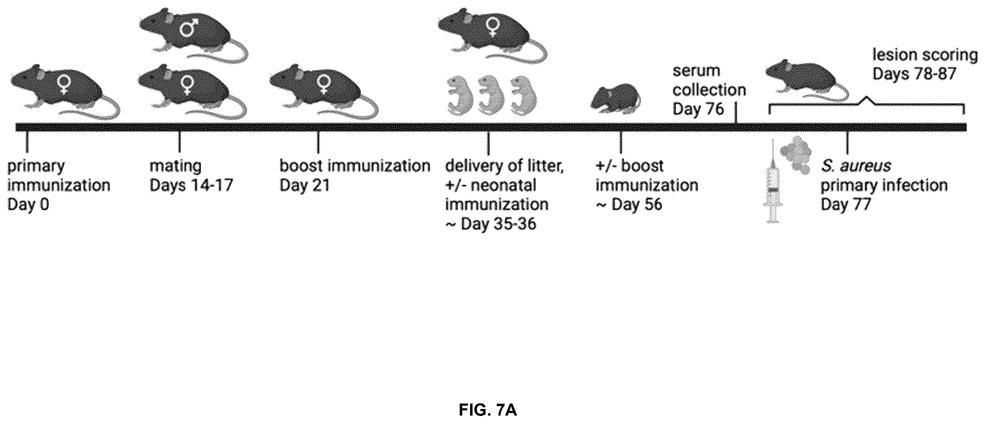

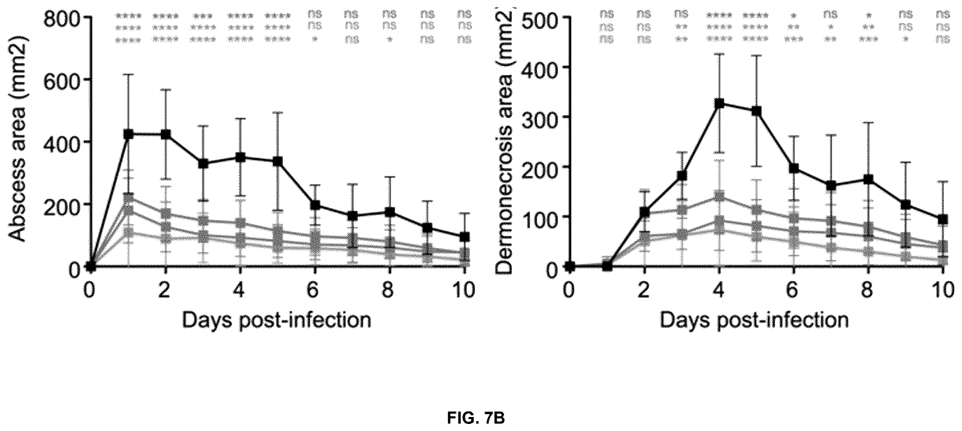

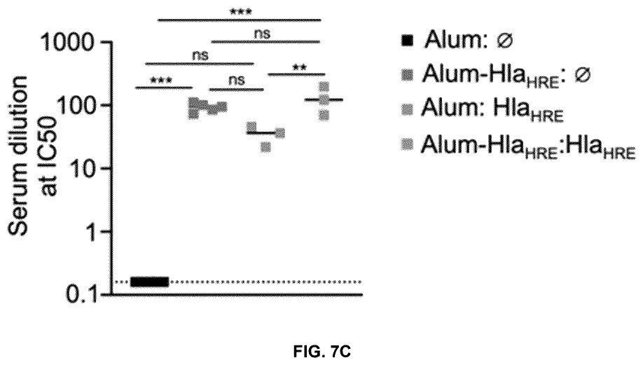

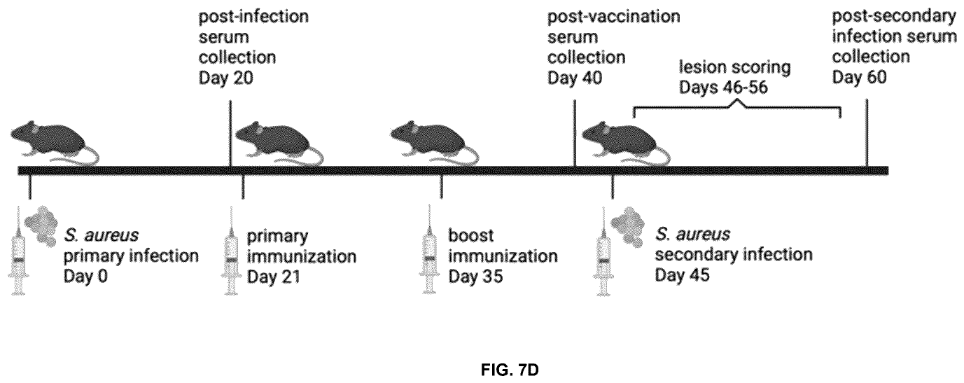

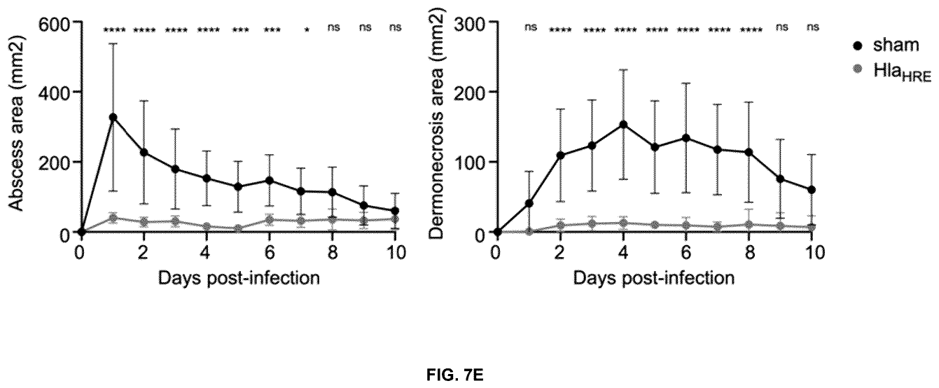

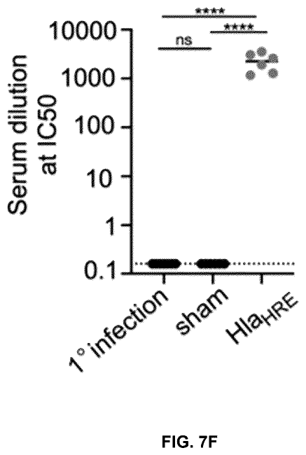

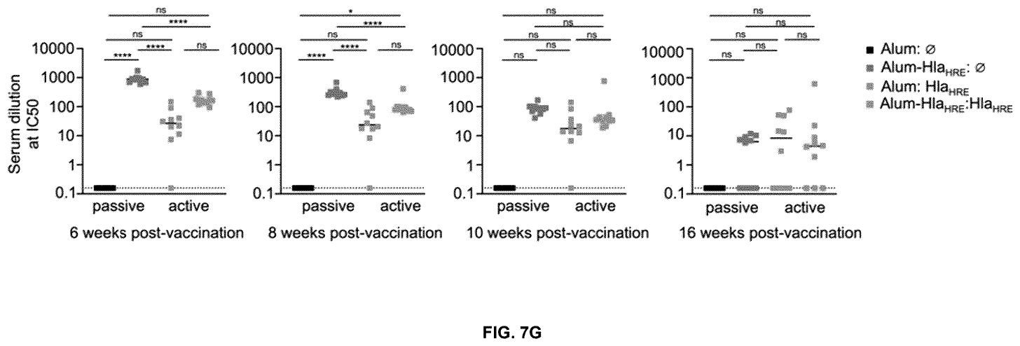

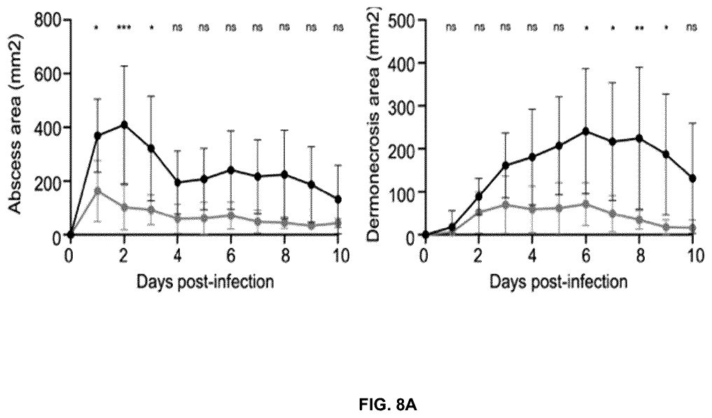

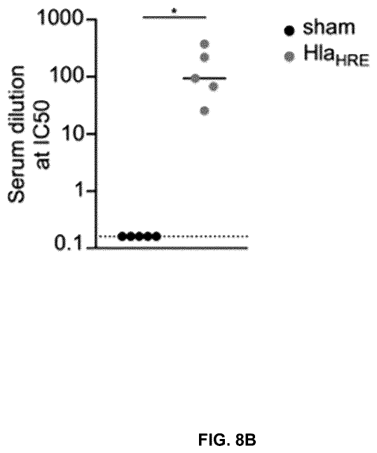

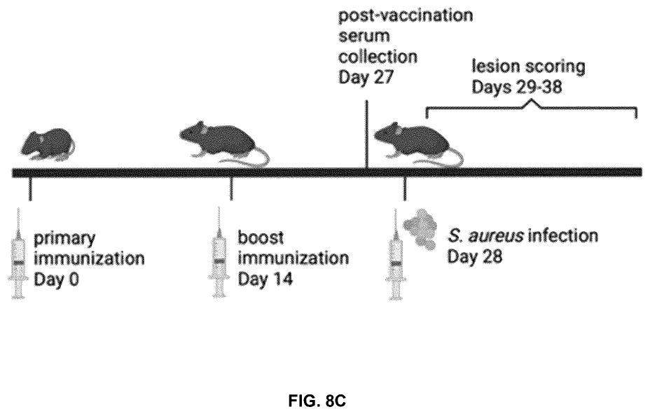

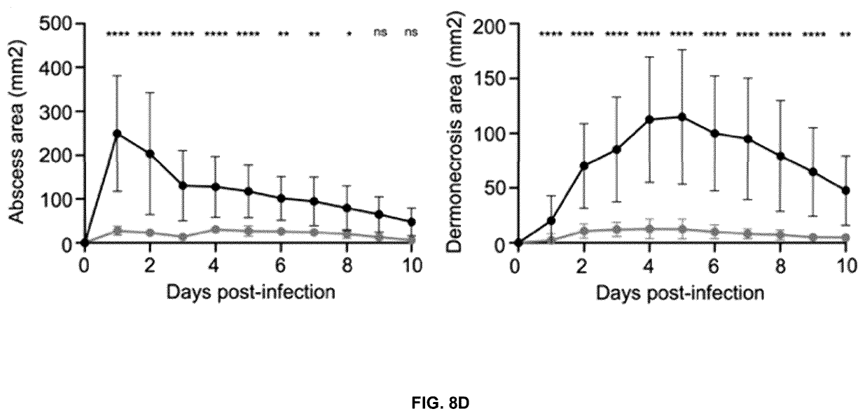

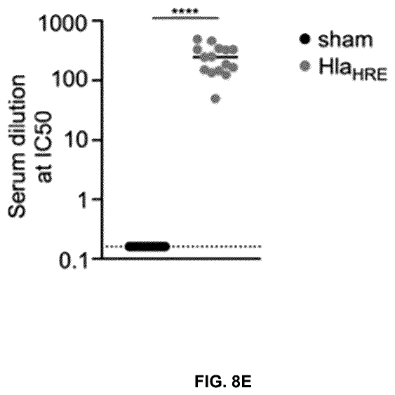

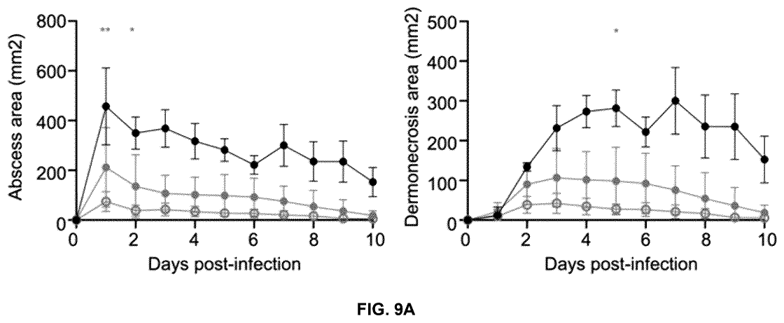

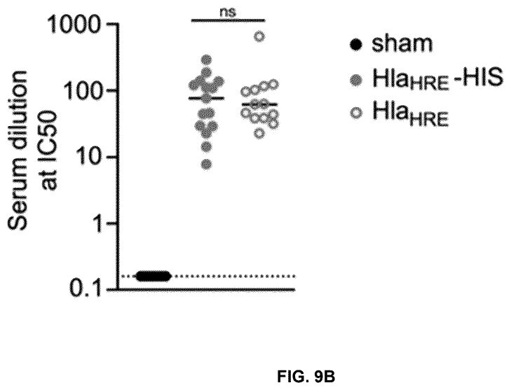

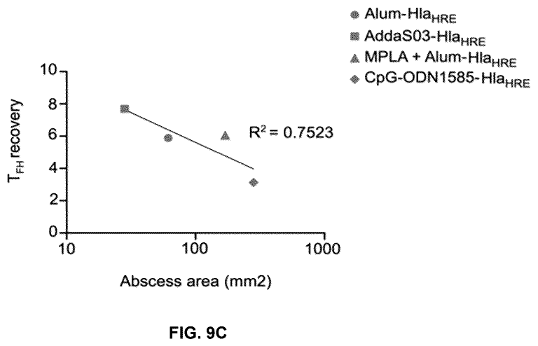

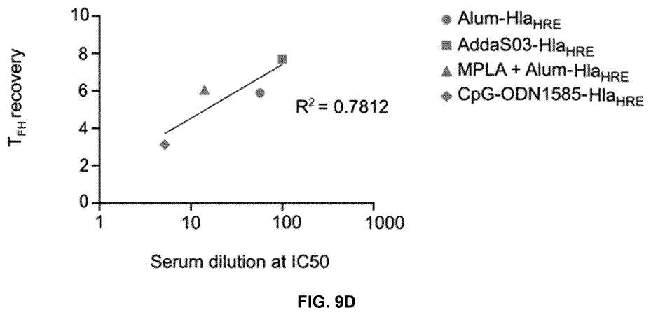

Each antigen was delivered to groups of mice to examine vaccine efficacy compared to Freunds adjuvant alone control. Data for skin abscess (left) and dermonecrosis (right) area following subcutaneous delivery of 1×10⁸ CFU S. aureus USA300/LAC strain to mice (9-10 per group) immunized with each of the Hla variant vaccines is provided. FIG. 3D provides data to show protection against rRBC lysis elicited by candidate vaccine antigens Hla_{H35L} (HlaH, purple), and Hla_{D45A/Y118F}, and Hla_{R66C/E70C} following incorporation of the fully detoxifying histidine 35 to leucine mutation (Hla_{H35UD45A/Y118F} and Hla_{H35UR66C/E70C}, Hla_{HDY} and Hla_{HRE}, respectively).

FIG. 4A-4H provide analysis of Hla-targeting vaccine candidate antigens. FIG. 4A provides OT-II CD4⁺ T cell subset analysis of OT-II CD4⁺ T cells harvested from the draining lymph nodes of mice immunized with adjuvant alone (sham) or candidate Hla vaccines Hla_{H35L}, Hla_{D45A/Y118F}, Hla_{R66C/E70C}. Tissue harvest occurred 7 days following subcutaneous infection with 1×10⁸ CFU of wildtype S. aureus USA300/LAC strain, p<0.05 by non-parametric 1-way ANOVA with Bonferroni-Dunn correction. FIG. 4B shows the correlation between protection against rRBC lysis upon treatment with 2 nM Hla and serologic anti-Hla titer (Log EC50) from mice following immunization with Hla_{H35L} (purple), Hla_{D45A/Y118F} (orange), Hla_{R66C/E70C} (blue). FIG. 4C is a comparison of vaccine antigen Hla variant binding to rRBC and wild-type Hla. FIG. 4D shows Melting curve analysis of Hla_{H}, Hla_{HDY}, and Hla_{HRE} compared to wild-type Hla. FIG. 4E is an SDS-PAGE analysis of ³⁵S-methionine labelled Hla, Hla_{H}, Hla_{HDY}, and Hla_{HRE} produced by coupled in vitro transcription and translation. FIG. 4F shows analysis of heptamer assembly and stability for Hla, Hla_{H}, Hla_{HDY}, and Hla_{HRE}, evaluated following incubation of radiolabeled toxin with rRBCs for one hour at room temperature followed by incubation at 37° C., 62° C., or 80° C. for 10 minutes. FIG. 4G shows analysis of Hla_{HRE} heptamer capability with increasing concentrations of radiolabeled toxin. Radiolabeled toxin was incubated with rRBCs for one hour at room temperature followed by incubation at 37° C. Relative densitometric units of monomeric and heptameric toxin was quantified using ImageJ software. SDS-PAGE separation was followed by phosphor-image detection of labeled toxin; for FIGS. 4E-4G, toxin monomers (arrow) and oligomers (Hla₇) are noted. FIG. 4H shows disulfide bond identified in antigen Hla_{HRE} by mass spectrometry (mass spectrometry peptide sequence provided in SEQ ID NO:. 47) Disulfide bond (S—S) peptide linking of Cys66 and Cys70 within the Hla_{HRE} protein was identified by LC-MS/MS analysis and validated by manual alignment of y or b-ions matching.

FIG. 5A-5E show that neonatal vaccination provide protection against S. aureus skin and soft tissue infection. FIG. 5A is a schematic showing timeline of neonatal vaccination and SSTI modeling. FIG. 5B provides graphs showing skin abscess (left) area following re-infection of immunized mice (9-10 per group) with 1×10⁸ CFU S. aureus. *p<0.05, **p<0.01, ***p<0.001, ****p<0.0001, noting statistical significance of Hla_{HRE} compared to each vaccine condition designated by color and dermonecrosis (right) area following re-infection of immunized mice (9-10 per group) with 1×10⁸ CFU S. aureus. *p<0.05, **p<0.01, ***p<0.001, ****p<0.0001, noting statistical significance of Hla_{HRE}compared to each vaccine condition designated by color. FIG. 5C provides graphs showing skin abscess (left) area following re-infection of immunized mice (9-10 per group) with 1×10⁸ CFU S. aureus. *p<0.05, **p<0.01, ***p<0.001, ****p<0.0001, noting statistical significance of Hla_{HRE} compared to each vaccine condition designated by color and dermonecrosis (right) area following re-infection of immunized mice (9-10 per group) with 1×10⁸ CFU S. aureus. *p<0.05, **p<0.01, ***p<0.001, ****p<0.0001, noting statistical significance of Hla_{HRE} compared to each vaccine condition designated by color. FIG. 5D shows serum dilution from mice in FIG. 5B and C that affords 50% protection against rRBC lysis (IC50) when exposed to 0.8pM recombinant Hla. FIG. 5E shows correlation between serum dilution that affords 50% protection against Hla-mediated (0.8pM) rRBC lysis and anti-Hla titer from mice following immunization.

FIG. 5F-5H show that Hla_{HRE} vaccine amplifies protective immunity to S. aureus infection independent of the vaccine adjuvant. Mice received a priming vaccine with 48 hours of birth and a boost vaccine at the time of weaning, then were infected at 5-6 weeks of age for primary infection, followed by re-infection at 21 days on the contralateral flank as shown. FIG. 5F provides data for skin abscess (left) and dermonecrosis (right) area following re-infection of mice immunized with AddaS03 and the Hla polypeptide, via subcutaneous delivery of 1×10⁸ CFU S. aureus USA300/LAC strain. Mice were immunized with each of the following Hla variant vaccines: Hla_{H} (purple), Hla_{HDY} (orange), Hla_{HRE} (blue) or adjuvant-only control (black). FIG. 5G provides data for skin abscess (left) and dermonecrosis (right) area following re-infection of mice immunized with MPLA+Alum and the Hla polypeptide, via subcutaneous delivery of 1×10⁸ CFU S. aureus USA300/LAC strain. Mice were immunized with each of the following Hla variant vaccines: Hla_{H} (purple), Hla_{HDY} (orange), Hla_{HRE} (blue) or adjuvant-only control (black). FIG. 5H provides data for skin abscess (left) and dermonecrosis (right) area following re-infection of mice immunized with CpG-ODN1585 and the Hla polypeptide, via subcutaneous delivery of 1×10⁸CFU S. aureus USA300/LAC strain. Mice were immunized with each of the following Hla variant vaccines: Hla_{H} (purple), Hla_{HDY} (orange), Hla_{HRE} (blue) or adjuvant-only control (black). *p<0.05, **p<0.01, ***p<0.001, ****p<0.0001, calculated by two-way ANOVA and Tukey test for multiple comparisons, noting statistical significance of Hla_{HRE} compared to each vaccine condition designated by legend color.

FIG. 6A-6K show that Hla_{HRE} amplifies the host T_{FH} to confer augment protective immunity. FIG. 6A shows the serum dilution from immunized mice that affords 50% protection against rRBC lysis (IC50) when exposed to 0.8pM recombinant Hla (sera collected 1 week or 3 weeks post-boost). FIG. 6B shows analysis of serum-mediated neutralization of Hla binding to rRBC comparing immune sera collected 1 week post boost vaccination from mice. FIG. 6C provides densitometric analysis of Hla oligomer formation on rRBC comparing immunized sera from mice in FIG. 6A and FIG. 6B. FIG. 6D provides analysis of inguinal germinal center cells 1 week following immunization of mice with either the Hla_{H} or Hla_{HRE} vaccines comparing GL7⁺Fas⁺B median fluorescence intensity (MFI) on CD4⁺ T cells. FIG. 6E provides analysis of inguinal germinal center cells 1 week following immunization of mice with either the Hla_{H} or Hla_{HRE}vaccines comparing Ki67⁺GL7⁺Fas⁺B cells median fluorescence intensity (MFI) on CD4⁺ T cells. FIG. 6F provides analysis of inguinal germinal center cells 1 week following immunization of mice with either the Hla_{H} or Hla_{HRE} vaccines comparing CD4⁺ T cells median fluorescence intensity (MFI) on CD4⁺ T cells. FIG. 6H provides analysis of inguinal germinal center cells 1 week following immunization of mice with either the Hla_{H} or Hla_{HRE} vaccines comparing CXCR5 median fluorescence intensity (MFI) on CD4⁺ T cells. FIG. 6I provides photographs showing gross pathology of skin lesions in groups of mice 2 days following secondary infection with S. aureus. FIG. 6J provides analysis of CXCR5^{hi}Bcl6⁺T_{FH} cell compartment in mice. FIG. 6K provides analysis of CXCR5^{hi}Bcl6⁺T_{FH} cell compartment in mice subjected to primary infection with wild-type (WT) S. aureus, the Δhla isogenic variant, or the Δhla variant complemented with a plasmid that enables restoration of Hla expression (Δhla::phla). *p<0.05, **p<0.01, ***p<0.001, ****p<0.0001

FIG. 6L-M show that maternal antibody protection diminishes after 16 weeks post vaccination with Hla_{HRE}. FIG. 6L provides flow-cytometry data with T_{FH} cell population marked as CXCR5⁺ICOS⁺PD1⁺ generated in response to re-infection of immunized mice with 1×10⁸ S. aureus. FIG. 6M provides flow-cytometry data with T_{FH} population generated following infection of mice with 1×10⁸ S. aureus USA300/LAC or its Δhla isogenic variant.

FIG. 7A-7G show that Hla_{HRE} vaccine remains effective after maternal vaccination and pre-exposure to S. aureus SSTI infection. FIG. 7A is a schematic showing timeline of neonatal vaccination and SSTI modeling. FIG. 7B provides graphs showing skin abscess (left) and dermonecrosis (right) area of mice (4-8 per group) following primary infection with 1×10⁸ CFU S. aureus. Mice were born to dams that received Alum or the Alum-Hla_{HRE} vaccine; pups were either unimmunized (black and blue, respectively), Alum immunized (gray), or Alum-Hla_{HRE} immunized (green). *p<0.05, **p<0.01, ***p<0.001, ****p<0.0001, noting statistical significance of Alum maternal immunization compared to each vaccine condition designated by color. FIG. 7C shows serum dilution from mice in FIG. 7B, that affords 50% protection against rRBC lysis (IC₅₀) when exposed to 0.8pM recombinant Hla FIG. 7D is a pre-exposure schematic showing timeline of primary infection, immunization schedule and secondary infection of juvenile mice. FIG. 7E provides graphs showing skin abscess (left) and dermonecrosis (right) area of mice (13-14 per group) following secondary infection with 1×10⁸ CFU S. aureus. FIG. 7F shows serum dilution from mice in FIG. 7E, that affords 50% protection against rRBC lysis (IC50) when exposed to 0.8pM recombinant Hla. FIG. 7G provides serum dilutions that affords 50% protection against rRBC lysis (IC50) when exposed to 0.8pM recombinant Hla with Alum (black) or Alum-Hla_{HRE}(blue) without subsequent active immunization of offspring (designated ø), or following maternal immunization with Alum or Alum-Hla_{HRE} followed by subsequent active immunization of offspring with Alum-Hla_{HRE} (gray and green, respectively). Pre-infection sera was collected at 6, 8, 10 and 16 weeks to analyze maternal antibody longevity. *p<0.05, **p<0.01, ***p<0.001, ****p<0.0001, calculated by one-way ANOVA and Tukey test for multiple comparisons.

FIG. 8A-8E show that vaccination with Hla_{HRE} demonstrates efficacy in distinct biological settings. FIG. 8A provides graphs showing skin abscess (left) and dermonecrosis (right) area following infection of immunized female mice (6-8 per group) with 1×10⁸ CFU S. aureus. *p<0.05, **p<0.01, ***p<0.001, ****p<0.0001. FIG. 8B provides serum dilution that that affords 50% protection against rRBC lysis (IC50) when exposed to 0.8pM recombinant Hla from mice in FIG. 8A. FIG. 8C is a schematic of juvenile vaccination schedule and SSTI infection. FIG. 8D provides graphs showing Skin abscess (left) and dermonecrosis (right) area following infection of immunized juvenile mice (10 per group) with 1×10⁸ CFU S. aureus. *p<0.05, **p<0.01, ***p<0.001, ****p<0.0001. FIG. 8E provide serum dilution that that affords 50% protection against rRBC lysis (IC50) when exposed to 0.8pM recombinant Hla from mice in FIG. 8D.

FIG. 9A-9D show that Hla_{HRE} vaccine remains effective against S. aureus SSTI infection and adjuvant formulations demonstrate differing T_{FH} responses. FIG. 9A provides graphs showing skin abscess (left) and dermonecrosis (right) area of mice (4-8 per group) following infection with 1×10⁸CFU S. aureus. *p<0.05, **p<0.01, noting statistical significance of histidine tagged vaccine compared to untagged Hla_{HRE} vaccine. FIG. 9B shows serum dilution from mice in FIG. 9A, that affords 50% protection against rRBC lysis (IC50) when exposed to 0.8pM recombinant Hla. FIG. 9C is a graph showing the inverse correlation of T_{FH} cell recovery and clinical abscess size from mice after S. aureus SSTI infection. FIG. 9D is a graph showing direct correlation between the anti-Hla neutralizing IC50 and T_{FH} response from mice after S. aureus SSTI infection.

The drawing figures do not limit the present inventive concept to the specific aspects disclosed and described herein. The drawings are not necessarily to scale, emphasis instead being placed on clearly illustrating principles of certain aspects of the present inventive concept.

### DETAILED DESCRIPTION

The following detailed description references the accompanying drawings that illustrate various aspects of the present disclosure. The drawings and description are intended to describe aspects and aspects of the present disclosure in sufficient detail to enable those skilled in the art to practice the present disclosure. Other components can be utilized, and changes can be made without departing from the scope of the present disclosure. The following description is, therefore, not to be taken in a limiting sense.

The present disclosure is based in part on the surprising discovery that certain rationally designed modifications in the context of previously characterized detoxified α-Hemolysin (Hla) antigens with disrupted pore forming capabilities, can effectively activate the germinal center (GC) and T follicular helper (TFH) cell response thus effecting long term immunity against S. aureus. Some pore forming mutations in Hla have been previously characterized and detailed description of these mutants and efficacy of antigenic Hla comprising these mutations can be found in PCT/US2020/066463, the disclosures of which is herein incorporated by reference in its entirety.

With extensive experimentation and thoughtful design, the current disclosure provides substitution mutations, in the context of the previously characterized detoxified α-Hemolysin (Hla) with a substitution at the H35 position, that provide robust immunologic protection coupled with the substantial increase in functional neutralizing response. It was highly unexpected that two amino acid substitutions R66C and E70C relative to Hla_{H35L} would markedly alter the outcome of vaccination. These modifications (Hla H35L/R66C/E70C, hereby referred to as Hla_{HRE} perform better than the previously characterized mutants as vaccines. Data presented herein show that they impact the GC and TFH response. The GC is a specialized microstructure that forms in secondary lymphoid tissues and is known to provide long-lived antibody secreting plasma cells and memory B cells, thus eliciting protection against reinfection. GC formation is critically dependent on TFH cells. In the GCs, B-cells undergo somatic mutation of the genes encoding their B cell receptors which, following successful selection, can lead to the emergence of B cell clones that bind antigen (for example Hla) with high affinity. In the context of Hla, high affinity antibodies can be neutralizing.

Staphylococcal α-hemolysin (Hla or α-toxin) is the founding member of a family of bacterial pore-forming 3-barrel toxins. It's structural gene, hla, is located on the chromosome of all S. aureus strains examined and encodes a precursor polypeptide that is 319 amino acids in length. The polypeptide is secreted as a 293-residue water-soluble monomer (amino acid sequence provided in SEQ ID NO: 1-mature Hla sequence). Studies of genetic variation in clinical isolates indicate significant Hla conservation across strains at both the nucleotide and protein level. Hla is thought to engage surface receptors of sensitive host cells, thereby promoting its oligomerization into a heptameric pre-pore and insertion of a β-barrel structure with 2 nm pore diameter into the plasma membrane. Hla pores form in lymphocytes, macrophages, alveolar epithelial cells, pulmonary endothelium and erythrocytes; however, granulocytes and fibroblasts appear less sensitive to overt lysis. Instillation of purified Hla into rabbit or rat lung tissue triggers vascular leakage and pulmonary hypertension, which has been attributed to release of several signaling molecules, e.g. phosphatidyl inositol, nitric oxide, prostanoids (PGE2, PGI2) and thromboxane A2. In agreement with the biochemical attributes of Hla, mutations that abrogate Hla expression in S. aureus severely attenuate virulence of the bacteria in the murine pneumonia model, skin infection model, and sepsis model.

Hla utilizes ADAM10 as a cellular receptor to inflict cytolytic injury and initiate ADAM10-mediated insults to target cells. While Hla is not required for S. aureus survival, this toxin is essential for pathogenesis in animal models of severe skin infection, pneumonia, sepsis, peritonitis, corneal infection, and central nervous system infection. The tissue tropism of Hla, the result of nearly ubiquitous cellular expression of ADAM10, renders this single toxin a very widely utilized virulence factor in the molecular pathogenesis of S. aureus disease and a prominent vaccine candidate. However, to be effective as vaccines Hla needs to be detoxified, such that it is unable to form pores, as pore formation is intrinsically injurious to the host cell.

In some aspects, the current disclosure provides compositions comprising polypeptides, or polynucleotides encoding polypeptides, with an amino acid sequence corresponding to an attenuated Hla polypeptide or an immunogenic variant, derivative or fragment thereof and method of producing and using the same for providing long lasting protection against S. aureus and other related bacterial infections.

### I. COMPOSITIONS

### Modified Hla Polypeptides and Derivatives

In some aspects, the current disclosure encompasses a modified α-hemolysin (Hla) polypeptide, or a derivative, or fragment thereof comprising a substitution of a histidine amino acid at position 35 relative to SEQ ID NO: 1, wherein position 35 is substituted with any other amino acid that results in abrogating functional pore formation by destabilizing the heptameric structure (as provided in SEQ ID NO: 3, wherein X is any amino acid); and at least an amino acid substitution R66C or E70C relative to SEQ ID NO: 1. In an aspect, the modified Hla polypeptide comprises the amino acid substitutions H35L, R66C, and E70C relative to SEQ ID NO: 1 (hereby referred to as Hla_{HRE}). In one example, the modified Hla polypeptide comprises the sequence set forth in SEQ ID NO: 11, or a derivative, or fragment thereof.

The modified Hla polypeptide may comprise the amino acid substitutions H35X and R66C, or H35X and E70C. The modified Hla polypeptide may comprise the amino acid substitutions H35L and R66C, or H35L and E70C. The modified Hla polypeptide may comprise the amino acid substitutions H35X, R66C and E70C. The modified Hla polypeptide may comprise the amino acid substitutions H35L, R66C and E70C. The modified Hla polypeptide may comprise the amino acid sequence as set forth in any one of SEQ ID NOs: 8-11, or a sequence at least about 80% about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical thereto, or a derivative, or fragment thereof.

The modified Hla polypeptide may further comprise one or more modifications relative to the amino acid sequence set forth in SEQ ID NO: 11. Thus, the modified Hla polypeptide may comprise the amino acid sequence as set forth in any one of SEQ ID NOs: 12-35, or a derivative, or fragment thereof. In some aspects, the current disclosure encompasses a polypeptide comprising an amino acid sequence with at least about 80% about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to any one of SEQ ID NOS: 12-35 or a derivative or fragment thereof.

Also contemplated herein are derivatives, or fragments of the modified Hla polypeptide. The term “derivative” or “variant,” and “fragment,” when used herein with reference to a polypeptide, refers to a polypeptide related to the modified Hla polypeptide as disclosed herein, for example either by amino acid sequence, structure (e.g., secondary and/or tertiary), activity (e.g., enzymatic activity) and/or function. Derivatives, variants and fragments of the polypeptide can comprise one or more amino acid variations (e.g., mutations, insertions, and deletions), truncations, modifications, or combinations thereof compared to a wildtype polypeptide. Individual residues can be deleted, or a number of contiguous amino acids can be deleted. A stop codon may be introduced (by substitution or insertion) into an encoding nucleic acid sequence to generate a truncated protein. Insertional mutants typically involve the addition of material at a non-terminal point in the polypeptide. This may include the insertion of one or more residues. Terminal additions, called fusion proteins, may also be generated. Substitutional variants or derivatives typically contain the exchange of one amino acid for another at one or more sites within the protein and may be designed to modulate one or more properties of the polypeptide, with or without the loss of other functions or properties as disclosed. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

The size of an Hla protein or polypeptide (wild-type or modified) may comprise, but is not limited to, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115,120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acids or greater, and any range derivable therein, or derivative thereof. It is contemplated that polypeptides may be mutated by truncation, rendering them shorter than their corresponding wild-type form, but also they might be altered by fusing or conjugating a heterologous protein sequence with a particular function (e.g., for targeting or localization, for enhanced immunogenicity, for purification purposes, etc.). The modified Hla polypeptide fragments may comprise 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119,120, 121, 122, 123, 124, 125, 126, 127, 128, 129, 130, 131, 132, 133, 134, 135, 136, 137, 138,139, 140, 141, 142, 143, 144, 145, 146, 147, 148, 149, 150, 151, 152, 153, 154, 155, 156, 157,158, 159, 160, 161, 162, 163, 164, 165, 166, 167, 168, 169, 170, 171, 172, 173, 174, 175, 176,177, 178, 179, 180, 181, 182, 183, 184,185, 186, 187, 188,189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, or more contiguous amino acids of the disclosed polypeptide. The modified Hla polypeptide according to the disclosure may be a single domain of Hla or multiple domains of Hla attached by a linker polypeptide.

The disclosed modified Hla polypeptide may further comprise amino molecules or non-amino acid thus forming a proteinaceous composition. As used herein, an “amino molecule” refers to any amino acid, amino acid derivative, or amino acid mimic known in the art. In certain aspects, the residues of the proteinaceous molecule are sequential, without any non-amino molecule interrupting the sequence of amino molecule residues. In other aspects, the sequence may comprise one or more non-amino molecule moieties. In particular aspects, the sequence of residues of the proteinaceous molecule may be interrupted by one or more non-amino molecule moieties. Accordingly, the term “proteinaceous composition” encompasses amino molecule sequences comprising at least one of the 20 common amino acids in naturally synthesized proteins, or at least one modified or unusual amino acid. Accordingly, the term “proteinaceous composition” encompasses amino molecule sequences comprising at least one of the 20 common amino acids in naturally synthesized proteins, or at least one modified or unusual amino acid. Proteinaceous compositions may be made by any technique known to those of skill in the art, including (i) the expression of proteins, polypeptides, or peptides through standard molecular biological techniques, (ii) the isolation of proteinaceous compounds from natural or recombinant sources (e.g., E. coli, insect cells, yeast or the like), or (iii) the chemical synthesis of proteinaceous materials. The nucleotide as well as the protein, polypeptide, and peptide sequences for various genes have been previously disclosed and may be found in the recognized computerized databases. One such database is the National Center for Biotechnology Information's GenBank and GenPept databases. The coding regions for these genes may be amplified and/or expressed using the techniques disclosed herein or as would be known to those of ordinary skill in the art.

Amino acid sequence variants of Hla are contemplated and can be substitutional, insertional, or deletion variants. A modification in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113,114, 115, 116, 117, 118, 119, 120, 121, 122, 123, 124, 125, 126, 127, 128, 129,130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167,168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, or more non-contiguous or contiguous amino acids of the polypeptide, as compared to wild-type. A Hla polypeptide from any staphylococcus species and strain are contemplated for use in methods of the disclosure.

Variants typically lack one or more residues of the native or wild-type protein. Individual residues can be deleted, or a number of contiguous amino acids can be deleted. A stop codon may be introduced (by substitution or insertion) into an encoding nucleic acid sequence to generate a truncated protein. Insertional mutants typically involve the addition of material at a non-terminal point in the polypeptide. This may include the insertion of one or more residues. Terminal additions, called fusion proteins, may also be generated.

Proteins of the disclosure may be recombinant and synthesized in vivo, or synthesized in vitro. Alternatively, the disclosed recombinant protein (modified Hla polypeptide) may be isolated from bacteria. It is also contemplated that a bacterium containing such a variant may be implemented in compositions and methods of the disclosure. Consequently, a protein need not be isolated.

The present disclosure also provides recombinant polynucleotides encoding the proteins, polypeptides, peptides of the disclosure. The nucleic acid sequences for wild-type Hla is set forth in SEQ ID NO: 2, and a nucleic acid sequence encoding any Hla variant, or modified Hla polypeptide as disclosed herein is contemplated. Methods of generating polynucleotide sequences encoding the disclosed polypeptides are known in the art and further described herein below.

### Polynucleotides

Disclosed herein is a polynucleotide sequence comprising a nucleic acid sequence encoding the modified Hla polypeptide, or derivative, or fragment thereof disclosed herein.

The polynucleotide may comprise a nucleic acid sequence encoding the amino acid sequence comprising the sequence set forth in any one of SEQ ID NOs: 6-11, or a fragment thereof, or a derivative thereof, or an amino acid sequence 80% about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical thereto.

As used in this application, the term “polynucleotide” refers to a nucleic acid molecule that either is recombinant or has been isolated free of total genomic nucleic acid. Included within the term “polynucleotide” are oligonucleotides, recombinant vectors, including, for example, plasmids, cosmids, phage, viruses, virus like particles (VLP) and the like. The current disclosure provides isolated nucleic acid segments and recombinant vectors that include within their sequence a contiguous nucleic acid sequence encoding any one of SEQ ID NO: 6-35 or a fragment thereof, or a derivative thereof, or an amino acid sequence 80% about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical thereto. Polynucleotides may further include, in certain aspects, regulatory sequences, isolated substantially away from their naturally occurring genes or protein encoding sequences. Polynucleotides may be RNA, DNA, analogs thereof, or a combination thereof.

In this respect, the term “gene,” “polynucleotide” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. It also is contemplated that a particular polypeptide from a given species may be encoded by nucleic acids containing natural variations that having slightly different nucleic acid sequences but, nonetheless, encode the same or substantially similar protein.

In some aspects, the disclosure provides isolated nucleic acid segments and recombinant vectors incorporating nucleic acid sequences that encode the disclosed modified Hla polyeptide or any variant or fragment thereof as provided herein. Thus, an isolated nucleic acid segment or vector containing a nucleic acid segment may encode, for example, the modified Hla polypeptide comprising a H35X substitution, wherein X is any suitable amino acid, a R66C or an E70C substitution and that is immunogenic. In some aspects, an isolated nucleic acid segment or vector containing a nucleic acid segment may encode, for example, Hla (H35L) polypeptide comprising a R66C or an E70C substitution and that is immunogenic.

The term “recombinant” may be used in conjunction with a polypeptide or the name of a specific polypeptide, and this generally refers to a polypeptide produced from a nucleic acid molecule that has been manipulated in vitro or that is a replication product of such a molecule. In other aspects, the disclosure concerns isolated nucleic acid segments and recombinant vectors incorporating nucleic acid sequences that encode the modified Hla polypeptide that can be used to generate an immune response in a subject. In various aspects the nucleic acids of the disclosure may be used in genetic vaccines.

The nucleic acid segments used in the present disclosure, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In some aspects, the nucleic acid used in the present disclosure encodes modified Hla polypeptides as disclosed herein or a derivative or fragment thereof. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein.

In some aspects, the term “functionally equivalent codon” is used herein to refer to codons that encode the same amino acid, such as the six codons for arginine or serine, and also refers to codons that encode biologically equivalent amino acids. It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in an amino acid sequence, and in its underlying DNA coding sequence, and nevertheless produce a protein with like properties. It is thus contemplated by the inventors that various changes may be made in the DNA sequences of genes or nucleic acids without appreciable loss of their biological utility or activity.

In some aspects, the current disclosure encompasses isolated polynucleotides, plasmids, viruses, virus like particles (VLP), viral vectors, cosmids, phage comprising a nucleic acid sequence encoding a polypeptide at least about 80% about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to any of SEQ ID NOS: 6-35 or a derivative or fragment thereof.

The nucleic acid sequences which encode the disclosed modified Hla polypeptide of the disclosure can be operatively linked to one or more regulatory sequences. Operatively linked refers to a juxtaposition wherein the components so described are in a relationship permitting them to function in their intended manner. An expression control sequence operatively linked to a coding sequence is achieved under conditions compatible with the expression control sequences. As used herein, the expression control sequences refer to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, and maintenance of the correct reading frame of that gene to permit proper translation of the mRNA and stop codons. The term “control sequences” is intended to include, at a minimum, components whose presence can influence expression, and can also include additional components whose presence is advantageous, for example, leader sequences and fusion partner sequences. Expression control sequences can include a promoter.

In some aspects, a vector comprising the nucleic acid sequence encoding the modified Hla can be a plasmid, cosmid, yeast artificial chromosome (YAC), bacterial artificial chromosome (BAC), viral vector or bacteriophage. The vectors can provide for replication of the nucleic acid sequence polynucleotide sequence encoding the modified Hla polypeptides, expression of Hla polypeptides or integration of Hla nucleic acids into the chromosome of a host cell. The choice of vector is dependent on the desired purpose. Certain cloning vectors are useful for cloning, mutation and manipulation of the Hla nucleic acid. Other vectors are useful for expression of the Hla polypeptide, being able to express the polypeptide in large amounts for purification purposes or to express the Hla polypeptide in a temporal or tissue specific manner. The vector can also be chosen on the basis of the host cell, e.g., to facilitate expression in bacteria, mammalian cells, insect cells, fish cell (e.g., zebrafish) and/or amphibian cells. The choice of matching vector to host cell is apparent to one of skill in the art, and the types of host cells are discussed below. Many vectors or vector systems are available commercially, for example, the pET bacterial expression system (Invitrogen™, Carlsbad Calif.).

The vectors disclosed herein can be viral or non-viral vectors. For example, as discussed above the disclosed vectors can be viral vectors. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain neurodegenerative diseases or disorders and cell populations by using the targeting characteristics of the carrier.

Vectors can include various components including, but not limited to, an origin of replication, one or more marker or selectable genes (e.g. GFP, neo), promoters, enhancers, terminators, poly-adenylation sequences, repressors or activators. Such elements are provided in the vector so as to be operably linked to the coding region of the Flla-encoding nucleic acid, thereby facilitating expression in a host cell of interest. Cloning and expression vectors can contain an origin of replication which allows the vector to replicate in the host cells. Vectors can also include a selectable marker, e.g., to confer a resistance to a drug or compliment complement deficiencies in growth. Examples of drug resistance markers include, but are not limited to, ampicillin, tetracycline, neomycin or methotrexate. Examples of other marker genes can be the fluorescent polypeptides such as one of the members of the fluorescent family of proteins, for example, GFP, YFP, BFP, RFP etc. These markers can be contained on the same vector as the gene of interest or can be on separate vectors and co transfected with the vector containing the gene of interest.

The vector can contain a promoter that is suitable for expression of the Hla in mammalian cells, which promoter can be operably linked to provide for inducible or constitutive expression of the disclosed modified Hla polypeptide. Exemplary inducible promoters include, for example, the metallothionine promoter or an ecdysone-responsive promoter. Exemplary constitutive promoters include, for example, the viral promoters from cytomegalovirus (CMV), Rous Sarcoma virus (RSV), Simian virus 40 (SV40), avian sarcoma virus, the beta-actin promoter and the heat-shock promoters. The promoter can be chosen for its tissue specificity. Certain promoters only express in certain tissues, and when it is desirable to express the polypeptide of interest only in a selected tissue, one of these promoters can be used. The choice of promoter will be apparent to one of skill in the art for the desired host cell system.

The vector encoding the modified Hla polypeptide can be a viral vector. Examples of viral vectors include retroviral vectors, such as: adenovirus, adenoassociated virus (AAV), simian virus 40 (SV40), cytomegalovirus (CMV), Moloney murine leukemia virus (MoMuLv), Rous Sarcoma Virus (RSV), lentivirus, herpesvirus, poxvirus and vaccinia virus. A viral vector can be used to facilitate expression in a target cell, e.g., for production of the modified Hla polypeptide or for use in therapy (e.g., to deliver the modified Hla polypeptide to a subject by expression from the vector). Where used for therapy, Hla-encoding vectors (e.g, viral vectors), can be administered directly to the patient via an appropriate route or can be administered using an ex vivo strategy using subject cells (autologous) or allogeneic cells, which are suitable for administration to the patient to be treated. In some aspects, the vector encoding the modified Hla polypeptide can be a virus like particle or VLP. The term VLP is used in the general term as known in the art and corresponds to a small particle that contains certain proteins from the outer coat of a virus. Virus-like particles do not contain any genetic material from the virus and cannot cause an infection.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some aspects the nucleic acid sequences disclosed herein are derived from any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. The viral vectors may be formulated in pharmaceutical compositions as those described above

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; the teachings of which are incorporated herein by reference in their entirety for their teaching of methods for using retroviral vectors for gene therapy.

The disclosure provides an adeno-associated virus (AAV) vector which comprises, consists essentially of, or consists of a nucleic acid sequence encoding the disclosed modified Hla polypeptide. When the AAV vector consists essentially of a nucleic acid sequence encoding the disclosed modified Hla polypeptide, additional components can be included that do not materially affect the AAV vector (e.g., genetic elements such as poly(A) sequences or restriction enzyme sites that facilitate manipulation of the vector in vitro). When the AAV vector consists of a nucleic acid sequence encoding polypeptide, the AAV vector does not comprise any additional components (i.e., components that are not endogenous to AAV and are not required to effect expression of the nucleic acid sequence to thereby provide the Hla).

Other useful systems include, for example, replicating and host-restricted non-replicating vaccinia virus vectors. In addition, the disclosed nucleic acid sequences can be delivered to a target cell in a non-nucleic acid-based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Suitable methods for nucleic acid delivery to effect expression of compositions of the present disclosure are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by PEG mediated transformation of protoplasts (Omirulleh et al., 1993; U.S. Pat. Nos. 4,684,611 and 4,952,500, each incorporated herein by reference); or by desiccation/inhibition mediated DNA uptake (Potrykus et al., 1985). Through the application of techniques such as these, organelle(s), cell(s), tissue(s) or organism(s) may be stably or transiently transformed.

Thus, the polynucleotide compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ. Regarding liposomes, see, e.g., Brigham et al. Am. J. Resp. Cell. Mol. Biol. 1:95-100 (1989); Feigner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

Host cells modified to provide for expression of the disclosed modified Hla polypeptide are also contemplated. Such host cells can be modified to express the Hla polypeptide from either an episomal or genomically integrated nucleic acid. Such host cells can be produced by any suitable method, e.g., electroporation, transfection or transformation with a vector encoding the disclosed modified Hla polypeptide. Host cells can be selected according to a desired use (e.g., mammalian cell expression), and modified to provide for Hla expression according to methods well known in the art. Techniques for introducing the vectors into host cells and subsequent culture of the host cells are well known in the art.

Host cells (e.g., mammalian host cells) suitable for replication and expression of Hla containing vectors are provided, wherein the cells may be stably or transiently transfected and/or stably or transiently express the disclosed modified Hla polypeptide. Such Hla-expressing mammalian cells find use in, for example, production of the modified Hla polypeptide. Production of Hla in mammalian cells can provide for post-translational modifications of the Hla and/or to heterologous amino acids to which it may be fused (e.g., glycosylation, cleavage of signal peptide (if present)). In addition, mammalian cell lines can be selected for use in replicating, packaging and producing high titers of virus particles which contain the disclosed modified Hla polypeptide of interest or nucleic acid-encoding the disclosed modified Hla polypeptide. Such Hla containing viruses can then be used to provide for delivery of Hla-encoding nucleic acids and Hla polypeptides to a subject in need thereof.

Exemplary host cells include bacteria, yeast, mammalian cells (e.g., human cells or cell lines), insect cells, and the like. Examples of bacterial host cells include E. coli and other bacteria which can find use in cloning, manipulation and production of Hla nucleic acids or the production of Hla polypeptide. Examples of mammalian cells include, but are not limited to, Chinese hamster ovary (CHO) cells, HEK 293 cells, human cervical carcinoma cells (Hela), canine kidney cells (MDCK), human liver cells (HepG2), baby hamster kidney cells (BHK), and monkey kidney cells (CV1), Vero, CEM, 721.221, H9, Jurkat, Raji, W138, COS-7, 293, HepG2, 3T3, and RIN cell.

Immunogenic and/or Pharmaceutical Compositions

In some aspects, the current disclosure also encompasses immunogenic and pharmaceutical compositions comprising the modified Hla polypeptide or the polynucleotides encoding the modified Hla polypeptide disclosed herein and at least a pharmaceutically acceptable excipient.

The pharmaceutically acceptable excipient may be an adjuvant, a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science. In each of the aspects described herein, a composition of the disclosure may optionally comprise one or more additional drug or therapeutically active agent in addition to the Hla. Thus, in addition to the therapies described herein, one may also provide to the subject other therapies known to be efficacious for treatment of the disease, disorder, or condition.

In an aspect, the pharmaceutically acceptable excipient is an adjuvant. Any functional adjuvant that enhances the immunogenicity of the composition may be added to the Immunogenic and/or pharmaceutical composition. Non-limiting examples of adjuvants for use with the disclosed modified Hla polypeptides or polynucleotides include alum, Monophosphoryl lipid A (MPL), AddaSO3 (ASO3-like), MPLA+Alum (ASO4-like), CpG-ODN1585, ADJUPHOS®, aluminum hydroxide, Advax, MF9, IL-1, IL-2, IL-4, IL-7, IL-12, g-interferon, GMCSP, BCG, MDP compounds, such as thur-MDP and nor-MDP, CGP (MTP-PE), and lipid A, or any combination thereof.

In some aspects, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In some aspects, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In some aspects, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In some aspects, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In some aspects, the excipient may be a pH modifier. By way of non-limiting example, the pH modifying agent may be sodium carbonate, sodium bicarbonate, sodium citrate, citric acid, or phosphoric acid.

In some aspects, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In some aspects, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In some aspects, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In some aspects, the excipient may be a lubricant. Non limiting examples of suitable lubricants include minerals such as talc or silica; and fats such as vegetable stearin, magnesium stearate, or stearic acid.

In some aspects, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In some aspects, the excipient may be a flavoring agent. Flavoring agents may be chosen from synthetic flavor oils and flavoring aromatics and/or natural oils, extracts from plants, leaves, flowers, fruits, and combinations thereof.

In some aspects, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

### Administration

### Dosage Forms

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L, Eds., Pharmaceutical Dosage Forms, Marcel Dekker Inc., New York, N.Y. (1980). In a specific aspect, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including cutaneous, subcutaneous, intraocular, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some aspects, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In some aspects, a composition comprising the modified Hla polypeptides or modified Hla polynucleotides or variant thereof, may be encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present disclosure. Non limiting examples of suitable structured fluid delivery systems may include nanoparticles, liposomes, microemulsions, micelles, dendrimers, and other phospholipid-containing systems. Methods of incorporating compositions into delivery vehicles are known in the art.

In some aspects, a liposome delivery vehicle may be utilized. Liposomes, depending upon the aspect, are suitable for delivery of the modified Hla polypeptides or modified Hla polynucleotides, in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the composition comprising the Hla or variant thereof may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1 ′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the one or more of a tricyclic antipsychotic, vasodilator, antibiotic/antiseptic, aryl piperazine or derivatives thereof, may be prepared by any known method of preparing liposomes for drug delivery, such as, for example, detailed in U.S. Pat. Nos. 4,241,046; 4,394,448; 4,529,561 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred aspect the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of one or more of a proteotoxicity reducing agent or derivatives thereof, concentration and composition of lipid, concentration of multivalent cations, rate of mixing, presence of and concentration of solvent.

In some aspects, a composition of the disclosure may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the disclosure generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one aspect, the structure may be micelles, which are the simplest microemulsion structures that are generally spherical or cylindrical objects. Micelles are like drops of oil in water, and reverse micelles are like drops of water in oil. In an alternative aspect, the microemulsion structure is the lamellae. It comprises consecutive layers of water and oil separated by layers of surfactant. The “oil” of microemulsions optimally comprises phospholipids. Any of the phospholipids detailed above for liposomes are suitable for aspects directed to microemulsions. The one or more of a tricyclic antipsychotic, vasodilator, antibiotic/antiseptic, aryl piperazine or derivatives thereof may be encapsulated in a microemulsion by any method generally known in the art.

In some aspects, the Hla, may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome aspects are suitable for use in dendrimer aspects. Any method generally known in the art may be utilized to make dendrimers and to encapsulate compositions of the disclosure therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the disclosure. Generally, the size of dendrimers may range from about 1 nm to about 100 nm.

Generally, a safe and effective amount of the modified Hla composition is, for example, that amount that would cause the desired effect in a subject while minimizing undesired side effects. In various aspects, an effective amount of Hla described herein can substantially induce an immune response in a subject.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

In some aspects, there is between about 0.001 mg and about 10 mg of total polypeptide per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 pg/ml, mg/ml, or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% may be an Hla polypeptide as disclosed herein.

The present disclosure contemplates the administration of the modified Hla polypeptide or peptide to affect a preventative therapy against the development of a disease or condition associated with infection by a staphylococcus pathogen.

The present disclosure describes polypeptides, peptides, and proteins for use in various aspects of the present disclosure. For example, specific polypeptides are assayed for their abilities to elicit an immune response. In specific aspects, all or part of the proteins of the disclosure can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence which encodes a peptide of the disclosure is inserted into an expression vector, transformed or transfected into an appropriate host cell and cultivated under conditions suitable for expression. [00129]One aspect of the disclosure includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide described herein may be employed. The generation of recombinant expression vectors, and the elements included therein, are discussed herein. Alternatively, the protein to be produced may be an endogenous protein normally synthesized by the cell used for protein production.

Another aspect of the present disclosure uses autologous B lymphocyte cell lines, which are transfected with a viral vector that expresses an immunogen composition, and more specifically, a protein having immunogenic activity.

Other examples of mammalian host cell lines include, but are not limited to Vero and HeLa cells, other B- and T-cell lines, such as CEM, 721.221, H9, Jurkat, Raji, as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed.

A number of selection systems may be used including, but not limited to HSV thymidine kinase, hypoxanthine-guanine, phosphoribosyltransferase, and adenine phosphoribosyltransferase genes, in tk-, hgprt- or aprt-cells, respectively. Also, anti-metabolite resistance can be used as the basis of selection: dhfr, which confers resistance to trimethoprim and methotrexate; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large-scale production of cells and cell products. However, suspension cultured cells have limitations, such as tumorigenic potential and lower protein production than adherent cells.

The present disclosure includes methods for preventing or ameliorating staphylococcus infections. As such, the disclosure contemplates vaccines for use in both active and passive immunization aspects. Immunogenic compositions, proposed to be suitable for use as a vaccine, may be prepared most readily directly from immunogenic Hla polypeptide prepared in a manner disclosed herein. Preferably the antigenic material is extensively dialyzed to remove undesired small molecular weight molecules and/or lyophilized for more ready formulation into a desired vehicle. The disclosure includes compositions that can be used to induce an immune response against a polypeptide or peptide derived from the disclosed modified Hla polypeptide so as to protect against infection by a staphylococcus and against developing a condition or disease caused by such. In certain aspects a composition is formulated to be administered to a mucosal surface, e.g., an aerosol formulation.

Alternatively, other viable and important options for a protein/peptide-based vaccine involve introducing nucleic acids encoding the antigen(s) as DNA or mRNA vaccines. In this regard, recent reports described construction of recombinant vaccinia viruses expressing either 10 contiguous minimal CTL epitopes or a combination of B cell, CTL, and TH epitopes from several microbes and successful use of such constructs to immunize mice for priming protective immune responses. Thus, there is ample evidence in the literature for successful utilization of peptides, peptide-pulsed APCs, and peptide-encoding constructs for efficient in vivo priming of protective immune responses. The use of nucleic acid sequences as vaccines is exemplified in U.S. Pat. Nos. 5,958,895 and 5,620,896.

The preparation of vaccines that contain polypeptide or peptide sequence(s) as active ingredients is generally well understood in the art. Typically, such vaccines are prepared as injectables either as liquid solutions or suspensions: solid forms suitable for solution in or suspension in liquid prior to injection may also be prepared.

The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific aspects, vaccines are formulated with a combination of substances.

Vaccines may be conventionally administered parenterally, mucosally, intranasally, by inhalation, and/or by injection, for example, either subcutaneously or intramuscularly. Additional formulations which are suitable for other modes of administration include suppositories and, in some cases, oral formulations.

For suppositories, traditional binders and carriers may include, for example, polyalkalene glycols or triglycerides; such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The polypeptides and polypeptide-encoding DNA constructs may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, mucosally, intranasally, by inhalation, by injection and the like. The dosage of the vaccine will depend on the route of administration and will vary according to the size and health of the subject.

A given composition may vary in its immunogenicity. It is often necessary therefore to boost the host immune system, as may be achieved by coupling a peptide or polypeptide to a carrier. Exemplary and preferred carriers are keyhole limpet hemocyanin (KLH) and bovine serum albumin (BSA). Other albumins such as ovalbumin, mouse serum albumin, or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde, and bis-biazotized benzidine.

The immunogenicity of polypeptide or peptide compositions can be enhanced by the use of non-specific stimulators of the immune response, known as adjuvants. Suitable adjuvants include all acceptable immunostimulatory compounds, such as cytokines, toxins, or synthetic compositions. A number of adjuvants can be used to enhance an antibody response against the disclosed modified Hla polypeptide. Adjuvants can (1) trap the antigen in the body to cause a slow release; (2) attract cells involved in the immune response to the site of administration; (3) induce proliferation or activation of immune system cells; or (4) improve the spread of the antigen throughout the subject's body.

Adjuvants include, but are not limited to, oil-in-water emulsions, water-in-oil emulsions, mineral salts, polynucleotides, and natural substances. Specific adjuvants that may be used include alum, Monophosphoryl lipid A (MPL), AddaSO3 (AS03-like), MPLA+Alum (AS04-like), CpG-ODN1585, ADJUPHOS®, aluminum hydroxide, Advax, MF9 or any combination thereof. Other adjuvants that may be used include IL-1, IL-2, IL-4, IL-7, IL-12, g-interferon, GMCSP, BCG, aluminum hydroxide or other aluminum compound, MDP compounds, such as thur-MDP and nor-MDP, CGP (MTP-PE), and lipid A. RIBI, which contains three components extracted from bacteria, MPL, trehalose dimycolate (TDM), and cell wall skeleton (CWS) in a 2% squalene/Tween 80 emulsion. MHC antigens may even be used. Others adjuvants or methods are exemplified in U.S. Pat. Nos. 6,814,971, 5,084,269, 6, 656, 462, each of which is incorporated herein by reference).

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (Fluosol-DA®) used as a block substitute may also be employed to produce an adjuvant effect.

Exemplary, often preferred adjuvants include complete Freund's adjuvant (a non-specific stimulator of the immune response containing killed Mycobacterium tuberculosis), incomplete Freund's adjuvants, alum, AddaS03 (AS03-like), MPLA and alum (AS04-like), CpG-ODN1585 or any combination thereof. In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); low-dose Cyclophosphamide (CYP; 300 mg/m2) (Johnson/Mead, NJ) and cytokines such as y-interferon, IL-2, or IL-12 or genes encoding proteins.

In certain aspects, the present disclosure concerns compositions comprising one or more lipids associated with a nucleic acid or a polypeptide/peptide. A lipid is a substance that is insoluble in water and extractable with an organic solvent. Compounds other than those specifically described herein are understood by one of skill in the art as lipids, and are encompassed by the compositions and methods of the present disclosure. A lipid component and a non-lipid may be attached to one another, either covalently or non-covalently.

A nucleic acid molecule or a polypeptide/peptide, associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid or lipid-H la-associated composition of the present disclosure is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine (Gibco BRL)-poxvirus or Superfect (Qiagen)-poxvirus complex is also contemplated.

In certain aspects, a composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or any range there between, of a particular lipid, lipid type, or non-lipid component such as an adjuvant, antigen, peptide, polypeptide, sugar, nucleic acid or other material disclosed herein or as would be known to one of skill in the art. In a non-limiting example, a composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10% to about 35% phosphatidyl choline, and about 1% of a non-lipid component. Thus, it is contemplated that compositions of the present disclosure may comprise any of the lipids, lipid types or other components in any combination or percentage range.

The compositions and related methods of the present disclosure, particularly administration of the disclosed modified Hla polypeptide or Hla polynucleotide to a patient/subject, may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline, oxacillin, vancomycin or various combinations of antibiotics. In addition, administration of the disclosed modified Hla polypeptide or Hla polynucleotide or anti-Hla antibodies to a patient/subject may be used in combination with the administration of antivirulence agents, such as RIP.

In one aspect, it is contemplated that the Hla composition is used in conjunction with antibacterial and/or antivirulence treatment. Alternatively, the therapy may precede or follow the other agent treatment by intervals ranging from minutes to weeks. In aspects where the other agents and/or a proteins or polynucleotides are administered separately, one would generally ensure that a significant period of time did not expire between each delivery, such that the agent and the composition of the present disclosure would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

Various combinations may be employed, for example antibiotic therapy is “A” and the immunogenic molecule or antibody given as part of an immune or passive immune therapy regime, respectively, such as a Hla antigen, is “B”: A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A.

In some aspects, pharmaceutical compositions are administered to a subject. Different aspects of the present disclosure involve administering an effective amount of a composition to a subject. In some aspects of the present disclosure, the modified Hla polypeptide or polynucleotide may be administered to the patient to protect against infection by one or more staphylococcus pathogens. Alternatively, a nucleic acid sequence or expression vector comprising the same which encode one or more such polypeptides or peptides may be given to a subject as a preventative treatment. Additionally, such compounds can be administered in combination with an antibiotic and/or antivirulence agent. Such compositions will generally be dissolved or dispersed in a pharmaceutically acceptable carrier or aqueous medium.

The active compounds of the present disclosure can be formulated for parenteral administration, e.g., formulated for injection via the intravenous, intramuscular, sub-cutaneous, or even intraperitoneal routes. The preparation of an aqueous composition that contains a composition or compositions of the present disclosure will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Additional formulations of pharmaceutical delivery systems may be in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. A suitable pharmaceutically acceptable carrier to maintain optimum stability, shelf-life, efficacy, and function of the delivery system would be apparent to one of ordinary skill in the art. [00163]Controlled-release (or sustained-release) preparations may be formulated to extend the activity of the agent(s) reduce the dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Compositions described herein can also be used in combination with other therapeutic modalities, as described further below. Thus, in addition to the therapies described herein, one may also provide to the subject other therapies known to be efficacious for treatment of the disease, disorder, or condition.

### II. METHODS

In some aspects, the present disclosure provides a method for inducing an immune response against a bacterial pathogen, the method comprising; administering to a subject in need thereof, a polypeptide, a polynucleotide, an immunogenic composition or a pharmaceutical composition as described herein. Also provided herein are compositions for use in inducing an immune response and use of the compositions in the manufacture of a medicament for inducing an immune response.

The present disclosure provides the importance of mediating immunity to a bacterial pathogen (for example S. aureus) by eliciting a germinal center (GC) and T follicular helper (TFH) response in a subject in need thereof. In some aspects, the provides the importance of mediating immunity to a bacterial pathogen in early stages of life, for example in utero to within about 3 years. In some aspects, the present disclosure provides the importance of mediating immunity to a bacterial pathogen prior to a first infection by the pathogen. In some aspects, the current disclosure provides a method of inducing an immune response in a subject, in need thereof, the method comprising administration to the subject in need thereof a composition comprising the modified α-hemolysin (Hla) polypeptide or a derivative or fragment thereof, comprising amino acid substitutions H35L, R66C and E70C (Hla_{HRE}), relative to the amino acid sequence set forth in SEQ ID NO: 1. The modified Hla polypeptide of claim 1, comprising an amino acid sequence set forth in SEQ ID NO: 11, or a sequence at least about 80% identical thereto, or a fragment thereof. The disclosed method may comprise administering to the subject in need thereof, the polypeptide comprising an amino acid sequence as set forth in any one of SEQ ID NO: 6-11, or a derivative, or fragment thereof, or a polynucleotide encoding polypeptide as disclosed in SEQ ID NO: 6-11, or a derivative, or fragment thereof, or an immunogenic composition comprising the same.

As described herein, the methods generally comprise active immunization (administration) of infants and toddlers at the time of birth or thereafter within 3 years of life followed by booster immunizations during the primary series in infancy and childhood. For example, the methods as disclosed herein include methods for reducing or preventing a S. aureus infection in a subject, the methods generally comprising administering to the subject compositions as described herein at birth or shortly thereafter. In some aspects, at birth or shortly after birth may include but is not limited to, within about 1, 2, 3, 4, 5, 10, 15, 20, 25, 30, 35, 40, 45, 50, 55 minutes or more after birth. In some aspects, shortly after birth may include but is not limited to, within about 1, 2, 3, 4, 5, 6, 7, 8, 9, 10, 11, 12, 13, 14, 15, 16, 17, 18, 19, 20, 21, 22, 23, 24 hours or more after birth. In some aspects, shortly after birth includes within about 1, 2, 3, 4, 5, 6, 7 days or more after birth. In some aspects, shortly after birth includes within about 1, 2, 3, 4, 5, 6, 7, or 8 or more weeks after birth. In some aspects, the active immunization of infants and toddlers is within 1, 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36 months after birth. The disclosed method may comprise active immunization of a subject, wherein the subject may be a child, an adolescent or an adult by administering to the subject a composition as described herein. In some aspects, the subject may be administered the immunogenic composition at any age, for example less than 3 years of age, between about 3 years to about 10 years, or about 20 years to about 30 years. or about 30 years to about 40 years, or about 40 years to about 50 years, or about 50 years to about 60 years, or about 60 years to about 70 years, about 70 years to about 80 years, or about 80 years to about 90 years, or about 90 years to about 100 years or more of age.

The methods further comprise administering to the subject a composition as disclosed herein one or more times following the first administration. In some aspects, the one or more additional administrations include within about 1, 2, 3, 4, 5, 6, 7 days or more after the first administration. In some aspects, the one or more additional administrations include within about 1, 2, 3, 4, 5, 6, 7, 8, 9, 10, 11, or 12 months or more after the first administration. In some aspects, the one or more additional administrations include within about 1, 2, 3, 4, 5, 6, 7, 8, 9, 10, 11, or 12 years or more after the first administration.

In another aspect, the methods provide maternal immunization for neonatal protection, coupled with immunization during the primary series in infancy and early childhood. For example, the methods as disclosed herein include methods for reducing or preventing a S. aureus infection in a subject, the methods generally comprising administering to the mother of a subject, a composition as disclosed herein while the subject is in utero. In utero is a Latin term literally meaning “in the womb” or “in the uterus”. In some aspects, the methods elicit transplacental transfer of anti-Hla neutralizing antibodies. In some aspects, the mother is administered the compositions while in the second or third trimester of pregnancy. Thus, a mother can be administered the composition within about 13, 14, 15, 16, 17, 18, 19, 20, 21, 22, 23, 24, 25, 26, 27, 28, 29, 30, 31, 32, 33, 34, 35, 36, 37, 38, 39, or 40 weeks of pregnancy. The methods further comprise administering to the subject at birth or shortly after birth a composition as disclosed herein one or more times following the first administration to the mother while the subject was in utero.

In some aspects, the compositions of the current disclosure can be administered using any one oral, intranasal intravenous, intramuscular, sub-cutaneous, or even intraperitoneal routes.

In some aspects, the compositions of the current disclosure may be administered along with at least one additional immunogenic antigen is selected from a group consisting of a antigen selected from the group consisting of Opp3a, DItD, HtsA, LtaS, IsdA, IsdB IsdC, SdrC, SdrD, SdrE, SdrF, SdrG, SdrH, SrtA, SpA, Sbi, FmtB, beta-hemolysin, fibronectin-binding protein A (fnbA), fibronectin-binding protein B (fnbB), coagulase, Fig, map, Panton-Valentine leukocidin (pvl), alpha-toxin and its variants, gamma toxin (hlg) and variants, ica, immunodominant ABC transporter, Mg2+transporter, Ni ABC transporter, RAP, autolysin, laminin receptors, IsaA/PisA, IsaB/PisB, SPOIIIE, SsaA, EbpS, Sas A, SasF, SasH, EFB (FIB), SBI, Npase, EBP, bone sialo binding protein II, aureolysin precursor (AUR)/Sepp1, CNA, and fragments thereof such as M55, TSST-1, mecA, poly-N-acetylglucosamine (PNAG/dPNAG) exopolysaccharide, GehD, EbhA, EbhB, SSP-1, SSP-2, HBP, vitronectin binding protein, HarA, EsxA, EsxB, Enterotoxin A, Enterotoxin B, Enterotoxin C1, and novel autolysin

As discussed above, the disclosure concerns evoking an immune response in a subject against the disclosed modified Hla polypeptide or a variant or fragment thereof. In one aspect, the immune response can protect against or treat a subject having, suspected of having, or at risk of developing an infection or related disease. In some aspects, the method includes evoking an immune response in a subject prior to the subject's first exposure to S. aureus. In non-limiting examples, the immune response may be evoked in the subject in utero and/or at birth or within 3 years of birth or later.

In some aspects of the disclosure, compositions as disclosed herein generate an immune response in the subject thereby conferring protective immunity on a subject. Protective immunity refers to a body's ability to mount a specific immune response that protects the subject from developing a particular disease or condition that involves the agent against which there is an immune response. In an exemplary aspect, administration of the compositions disclosed herein generate a GC and TFH response. An immunogenically effective amount is an amount capable of conferring protective immunity to the subject.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, or polypeptide of the disclosure in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. In some aspects, the immune response refers to the generation of a GC response.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

A method of the present disclosure includes treatment for a disease or condition caused by a staphylococcus pathogen, as well as prevention of or reduction in infection so as to prevent or minimize the extent of exposure to the pathogen.

In some aspects, the treatment is administered in the presence of adjuvants or carriers in the absence or substantial absence of other staphylococcal antigens and/or proteins. Furthermore, in some examples, treatment comprises administration of other agents commonly used against bacterial infection, such as one or more antibiotics.

Administration of the disclosed modified Hla polypeptide or Hla polynucleotide or variant thereof can occur as a single event or over a time course of treatment. For example, one or more of the disclosed modified Hla polypeptide or Hla polynucleotide can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more. Compositions may be administered 1, 2, 3, 4, 5, 6, 7, 8, 9, 10, 11, 12, 13, 14, 15, 16, 17, 18, 19, 20 or more times, and/or they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range or combination derivable therein.

Without further elaboration, it is believed that one skilled in the art can, based on the above description, utilize the present disclosure to its fullest extent. The following specific aspects are, therefore, to be construed as merely illustrative, and not limitative of the remainder of the disclosure in any way whatsoever. All publications cited herein are incorporated by reference for the purposes or subject matter referenced herein.

As various changes could be made in the above-described materials and methods without departing from the scope of the disclosure, it is intended that all matter contained in the above description and in the examples given below, shall be interpreted as illustrative and not in a limiting sense.

### III. DEFINITIONS

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to 1%, such as less than or equal to ±0.5%, such as less than or equal to ±0.2%, such as less than or equal to 0.1%, such as less than or equal to ±0.05%.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

The terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B, and C.” An exception to this definition will occur only when a combination of elements, functions, steps or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a,” “an,” “the” and “said” are intended to mean that there are one or more of the elements. The terms “comprising,” “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues See, e.g., Batzer et al., Nucleic Acid Res. 19:5081 (1991), the disclosure of which is incorporated in its entirety herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

Within the context of the application a protein is represented by an amino acid sequence and correspondingly a nucleic acid molecule or a polynucleotide represented by a nucleic acid sequence. Identity and similarity between sequences: throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology,” “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [b162], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to lie or Val; Lys to Arg; Gln or Glu; Met to Leu or lie; Phe to Met, Leu or Tyr; Ser to Thr; Thr to Ser; Trp to Tyr; Tyr to Trp or Phe; and Val to lie or Leu.

As used herein, “operably linked” means that expression of a gene is under the control of a regulatory element with which it is spatially connected. In some instance the regulatory element is a promoter and when used in this context, the expression of a gene is under control of the promoter with which it is spatially connected. A regulatory element (e.g., a promoter) can be positioned 5′ (upstream) or 3′ (downstream) of a gene under its control. The distance between the regulatory element (e.g., promoter) and a gene can be approximately the same as the distance between that regulatory element (promoter) and the gene it controls in the gene from which the regulatory element (e.g., promoter) is derived. As is known in the art, variation in this distance can be accommodated without loss of function (i.e., without loss of promoter function).

As used herein, “regulatory elements” refer to any sequence elements that regulate, positively or negatively, the expression of an operably linked sequence. “Regulatory elements” include, without being limiting, a promoter, an enhancer, a leader, a transcription start site (TSS), a linker, 5′ and 3′ untranslated regions (UTRs), an intron, a polyadenylation signal, and a termination region or sequence, etc., that are suitable, necessary, or preferred for regulating or allowing expression of the gene or transcribable DNA sequence in a cell. Such additional regulatory element(s) can be optional and used to enhance or optimize expression of the gene or transcribable DNA sequence. A regulatory sequence can, for example, be inducible, non-inducible, constitutive, cell-cycle regulated, metabolically regulated, and the like. A regulatory sequence may be a promoter. As used herein, the term “promoter” refers to a DNA sequence that comprises an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences.

A “fragment” or “portion” of an amino acid sequence can be understood to mean an amino acid sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, or more amino acids) to a reference amino acid sequence and comprising, consisting essentially of, or consisting of an amino acid sequence of contiguous amino acids identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference amino acid sequence. Such an amino acid fragment or portion according to the disclosure can be, where appropriate, included in a larger amino acid sequence of which it is a constituent.

As used herein, an “isolated” refers to a biological component (such as a nucleic acid molecule, nucleic acid sequence, protein, or virus) that has been substantially separated or purified away from other biological components (e.g., other chromosomal and extra-chromosomal DNA and RNA, proteins and/or organelles). Nucleic acids, proteins, and/or viruses that have been “isolated” include nucleic acids, proteins, and viruses purified by standard purification methods. The term also embraces nucleic acids, proteins, and viruses prepared by recombinant expression in a host cell, as well as chemically synthesized nucleic acids or proteins. The term “isolated” (or purified) does not require absolute purity; rather, it is intended as a relative term. Thus, for example, an isolated or purified nucleic acid, protein, virus, or other active compound is one that is isolated in whole or in part from associated nucleic acids, proteins, and other contaminants. In an aspect, the term “substantially purified” refers to a nucleic acid, protein, virus or other active compound that has been isolated from a cell, cell culture medium, or other crude preparation and subjected to fractionation to remove various components of the initial preparation, such as proteins, cellular debris, and other components. In an aspect, isolated proteins or nucleic acids, or cells containing such, in some examples are at least 50% pure, such as at least 75%, at least 80%, at least 90%, at least 95%, at least 98%, or at least 100% pure. Isolated nucleic acid molecules may comprise one or more naturally occurring sequences, recombinant sequences, or combinations thereof. An isolated nucleic acid molecule may comprise any one or more of a gene, a transcription regulatory sequence, a translation regulatory sequence, coding sequence, non-coding sequence, plasmids, vector, or viral vector. The isolated nucleic acid molecule may also comprise one or more modified nucleotides.

### EXAMPLES

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

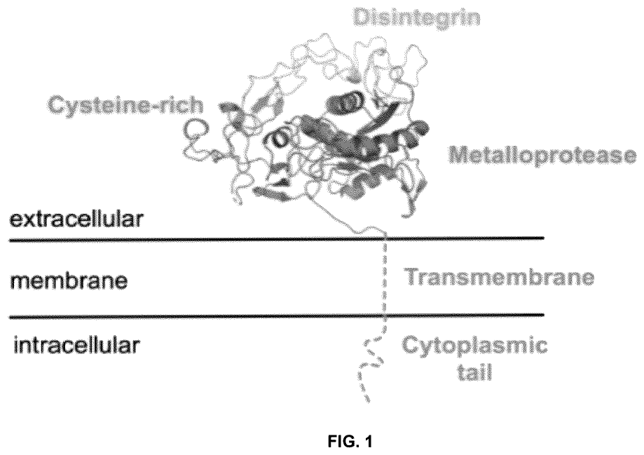

In some aspects, the current disclosure is a result of extensive intellectual and experimental efforts to identify an optimized non-toxic Hla variant immunogen capable of eliciting durable protective immunity against SSTI when vaccination is delivered in the neonatal period. S. aureus α-toxin (Hla) is highly conserved pore-forming toxin that contributes to pathogenesis of each of the leading manifestations of staphylococcal disease. Expressed by nearly all clinical isolates, Hla utilizes A Disintegrin and Metalloprotease 10 (ADAM10) as a cellular receptor to inflict host cell injury. ADAM10 is a type I transmembrane protein that is widely expressed on human cells (FIG. 1). Characterized by an N-terminal metalloprotease domain, ADAM10 contributes to cellular and tissue development, homeostasis, and response to injury, dependent on proteolytic cleavage of ADAM10 substrates in a cell-type specific manner. Upon binding of the Hla monomer to ADAM10, assembly of the heptameric toxin pore is initiated. Pore formation on the host cell membrane results in upregulation of the catalytic activity of ADAM10 and concomitant cleavage of native ADAM10 substrates including E-cadherin, VE-cadherin, and platelet glycoprotein VI. These cleavage events, together with cellular injury attributable to Hla pore formation, culminate in tissue-specific insults that are manifest in S. aureus disease.

Among staphylococcal virulence factors, Hla is unique in its ability to impair the antigen-specific T cell response, promoting recurrent infection. Data provided herein shows that the anti-Hla antibody titer is elevated among pediatric subjects who exhibit protection against recurrent infection, defining a correlate of human protective immunity. These findings suggest that eliciting a highly neutralizing anti-Hla antibody response early in life is a strategic target for vaccine development. A vaccine development approach was therefore designed to identify an optimized non-toxic Hla variant immunogen capable of eliciting durable protective immunity against SSTI when vaccination was delivered in the neonatal period. These development efforts were concentrated on the understanding of Hla-ADAM10 interaction and dramatic conformational changes that Hla undergoes to form the heptameric pore.

### Example 1: Analysis of Serologic Anti-Hla Titer by Age

Exposure to S. aureus occurs within the first weeks to months of life. Within the pediatric population, S. aureus skin and soft tissue infection (SSTI) is associated with disease recurrence in up to 50% of children. The anti-Hla antibody titer is elevated among children who exhibit protection against recurrent disease, defining a correlate of human protective immunity. These findings suggest that elicitation of a highly neutralizing antibody response to Hla early in life is a strategic target for vaccine-mediated protection against S. aureus. It has been shown that infants <1 year of age harbor a lower anti-Hla serologic titer and neutralizing capacity than individuals >2 years of age. To further resolve the influence of age on the generation of the anti-Hla response in the infant and pediatric population, a population of 343 subjects ranging in age from <1 to 18 years were evaluated.

TABLE 1
Characteristics of participants contributing serum samples

| Participant Characteristic | N (%) | |
|---|---|---|
| Age, years, median (range) | 8 | (0-18) |
| Age by category | | |

| | | | |
|---|---|---|---|
| <12 | mo | 22 | (6.4) |
| 12-23 | mo | 23 | (6.7) |
| 2-5 | yrs | 80 | (23.3) |
| 6-11 | yrs | 122 | (35.6) |
| 12-18 | yrs | 96 | (28.0) |

| | | |
|---|---|---|
| Hospitalized at time of study | 36 | (10.5) |
| enrollment | | |
| Taking antibiotics at time | 47 | (14.2) |
| of study enrollment | | |
| Colonized with S. aureus at | 138 | (40.2) |
| time of study enrollment | | |
| Comorbidities | | |
| Asthma | 58 | (17.6) |
| Allergies | 103 | (31.2) |
| Arthritis | 12 | (3.6) |
| Vasculitis | 1 | (0.3) |
| Immune system problem | 32 | (9.8) |
| Eczema | 54 | (16.4) |
| Seizures | 51 | (15.5) |
| Cancer | 147 | (49.2) |
| Sickle Cell | 7 | (2.1) |
| Inflammatory Bowel Disease | 23 | (7.1) |
| History of Staphylococcus | 17 | (5.3) |
| aureus infection | | |
| Participant experienced a skin | 38 | (11.5) |
| infection in the year prior to | | |
| study enrollment | | |
| Participant's household | 45 | (13.5) |
| member(s) experienced a | | |
| skin infection in the year | | |
| prior to study enrollment | | |
| Immunizations up-to-date | 308 | (93.3) |

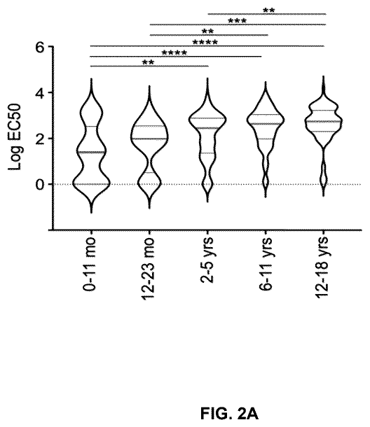

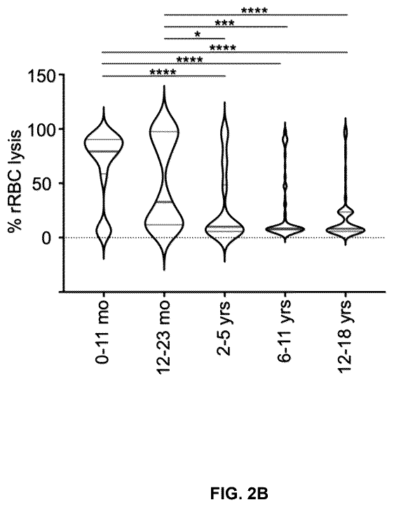

Analysis of the serologic anti-Hla titer by age revealed a gradual increase in the half-maximal effective concentration (EC₅₀) over the first five years of life, with titers children under 2 years of age being most distinct from those of older children (FIG. 2A). Analysis of the serum Hla neutralizing activity in a rabbit red cell (rRBC) protection assay confirmed that subjects <2 years exhibit response immaturity (FIG. 2B). S. aureus exposure through colonization is documented to occur in the first days and weeks of life. Indeed, the mortality from S. aureus infection over the first 4 years of life rivals that seen in older adults 1. Together, these data suggest that risk of both S. aureus disease and modification of the host response to this microbe occur early in life.

### Example 2: Development of Modified Non-Toxic Hla Variants

A vaccine development approach was designed to elicit immunity against S. aureus infection through neonatal vaccination. It was hypothesized that the Hla-ADAM10 interaction and subsequent conformational changes that induce pore formation may influence vaccine efficacy. A series of modified Hla antigens were designed based on structural determinants and correlates of toxin function, in part based on the expected immunogenic properties derived from a combination of prior observations and T cell epitope prediction algorithms. Six distinct antigens were generated and evaluated including three full-length Hla variant antigens appended with a 6x-histidine tag to facilitate affinity purification (FIG. 3A): Hla_{H35L} has been extensively studied as a toxoid vaccine that binds ADAM10 yet fails to form a stable pore, Hla_{D45A/Y118F}, predicted to interfere with b-barrel stem domain unfolding, and Hla_{R66C/E70C}, predicted to impair ADAM10 binding. A panel of peptide antigens (FIG. 3B): Hla50, harboring 5 tandem arrays of the first 50 amino acids of Hla that elicit protective immunity in animal models; HlaP1, a synthetic antigen harboring 5 tandem arrays of the Immune Epitope Database-predicted T cell epitope Hla36-50; and HlaP2, a synthetic antigen harboring three distinct predicted T cell epitopes from Hla (Hla36-50, Hla51-65, and Hla161-175) were also studied. Each antigen was screened by delivery to groups of mice to examine vaccine efficacy compared to Freund's adjuvant control. Upon a screening subcutaneous challenge with S. aureus (1×10⁸ colony forming units), mice receiving Hla_{H35L}, Hla_{D45A/Y118F}, and Hla_{R66C/E70C} vaccines exhibited protection against abscess formation and dermonecrosis (FIG. 3C). In contrast, mice receiving peptide vaccines exhibited similar or larger lesions than those observed in sham-vaccinated mice.

To further examine vaccine properties of the three full-length antigens, Hla_{D45A/Y118F} and Hla_{R66C/E70C} were modified to incorporate the H35L mutation to ensure full detoxification, which was verified in a rabbit red cell lysis assay (FIG. 3D, Hla_{H35L} denoted HlaH, HlaH35L/D45A/Y118F denoted Hla_{HDY}, and HlaH35L/R66C/E70C denoted Hla_{HRE}).

### Example 3: Characterization of Modified Non-Toxic Hla Variants

As an initial functional immunologic screen, each candidate antigen was delivered to mice in a prime-boost regimen formulated with Freund's adjuvants. Each candidate antigen enhanced the antigen-specific memory T cell response to infection, as indicated by utilizing the ovalbumin-specific OT-II T cell system and an ovalbumin-expressing S. aureus strain (FIG. 4A).

To assess the B cell response to immunization, the serum toxin-neutralizing capacity was determined in a rabbit red blood cell (rRBC) lysis protection assay and the half-maximal anti-Hla titer (EC50) in serum from vaccinated mice was quantified. The vaccine-elicited antibody response varied in an antigen-specific manner, with Hla_{HRE} eliciting a high degree of toxin neutralization (FIG. 4B). These findings suggest that nearly identical antigens based on primary sequence can exhibit distinct biological properties that modify antigenicity and host immune response.

To further characterize the biochemical properties of candidate antigens, ADAM10 binding was evaluated in a sensitive rRBC binding assay. Hla_{H} exhibited preservation of binding compared to wild-type Hla, with diminution of Hla_{HDY} binding and near-complete loss of Hla_{HRE} binding (FIG. 4C). Melt curve analysis of each antigen revealed that the Hla_{HDY} variant is structurally distinct from Hla, HlaH, and Hla_{HRE} (FIG. 4D). confirmed by SDS-PAGE analysis where the Hla_{HDY} variant formed the oligomeric toxin (Hla7) in solution without exposure to a cell membrane (FIG. 4E), while wild-type Hla and other variants were present solely in monomeric form (Hla1). The biological properties of these Hla variants on rRBCs were examined. It was observed that the oligomer formed by wild-type Hla exhibits temperature stability up to 65° C. (FIG. 4F left), illuminating the functional defect in the Hla_{H} variant that is unable to form a temperature-stable oligomer (FIG. 4F left). Hla_{HDY} retains the ability to oligomerize on the cell membrane proportionate to its binding capability (FIG. 4F right), whereas limited monomeric binding and oligomer formation were evident in the Hla_{HRE} variant (FIG. 4F right). To assess the oligomerization efficiency of Hla_{HRE} on the rRBC surface, increasing concentrations of Hla_{HRE} were tesed until achieving an equivalent amount of monomeric toxin rRBC binding as observed with Hla_{H} (FIG. 4G). Densitometric quantification of the oligomer:monomer ratio revealed a 3.6:1 ratio for Hla_{H} compared to 1.9:1 ratio for Hla_{HRE} signifying a functional oligomerization defect in addition to the binding defect observed in the Hla_{HRE} antigen. As Hla undergoes an ordered series of intramolecular movements upon binding to enable heptameric pore formation, the relative defect in functional oligomerization of Hla_{HRE} may represent broader conformational restriction in this antigen based on incorporation of a two cysteine residues that may enable disulfide bond formation. To examine whether Hla_{HRE} has a disulfide bond between C66 and E70, the antigen was subjected to mass spectroscopy-based analysis which revealed that the majority of purified Hla_{HRE} does incorporate an intramolecular disulfide bond (FIG. 4H).

### Example 4: Characterization of Vaccine Efficacy with Candidate Antigens

Each antigen's protective efficacy was evaluated in a prime-boost neonatal vaccination model (FIG. 5A), recognizing that neonates and infants represent the vaccine-targetable population in which to enhance the anti-Hla response in humans (FIGS. 2A and 2B). Several immunologic challenges arise in vaccine design for the neonatal population. The neonatal immune response is characterized by delayed formation of the germinal center (GC) with resultant decrease in the quantity and quality of the affinity-matured antibody response. Moreover, an increased frequency of FoxP3+T regulatory (TREG) cells in neonates, together with the presence of maternally derived antibodies that can modulate vaccine antigen availability and presentation, amplify the challenge of vaccine formulation for this population. An initial survey was performed of each candidate antigen formulated with four adjuvants: Alum, AddaSO3 (ASO3-like), MPLA+Alum (ASO4-like), and CpG-ODN1585. This array of adjuvants was selected for initial studies owing to their distinct immunostimulatory properties, reasoning that a broad-based approach to vaccine formulation would enable us to specifically evaluate the relevance of antigenic variation. To rigorously examine vaccine protection, a high inoculum of S. aureus was utilized (1×10⁸ CFU) to elicit primary SSTI in 5-6 week old mice with a secondary challenge performed on the contralateral flank 21 days following the primary infection (FIG. 5A). All antigens elicited protection against primary abscess formation (FIG. 5B left) and dermonecrosis (FIG. 5B right) when formulated with Alum. Upon recurrent infection, Hla_{HRE} elicited high-level protection against abscess formation and dermonecrosis (FIG. 5C) while Hla_{H} and Hla_{HDY} vaccines afforded limited protection. In the absence of re-boosting, the diminution of vaccine-mediated protection was prominent for Hla_{H} (76.9±9.3% to 28.4±11%) and Hla_{HDY} (76.1±13% to 11.5±4.6%) compared to that observed for Hla_{HRE} (94.4±2.6% to 76.5±8.3%). Enhanced protective immunity was observed with the Hla_{HRE} vaccine candidate during secondary infection irrespective of the adjuvant utilized (FIG. 5F, AddaSO3 (a), MPLA+Alum (b), CpG-ODN1585 (c)). Analysis of the anti-Hla neutralizing antibody titer in serum from each group of vaccinated mice prior to primary infection revealed a marked benefit in those mice receiving the Hla_{HRE} antigen irrespective of adjuvant (FIG. 5D).

Correlation analysis of the serologic anti-Hla antibody response and toxin neutralizing activity from each group of immunized mice reflected the importance of the vaccine antigen as the principal determinant of vaccine-mediated protection, with the Hla_{HRE} vaccine eliciting an increased anti-toxin neutralizing antibody response within each adjuvant group compared to the Hla_{H} vaccine (FIG. 5E FIG. 3e, Alum, 92.3-fold; AddaSO3, 23.8-fold; MPLA+Alum, 84.2-fold; CpG-ODN1585, 14.6-fold).

### Example 5: Characterization Of Protective Efficacy Of Hla_{HRE} In Mice

The degree of immunologic protection afforded by Hla_{HRE}, coupled with an increase in functional anti-toxin neutralizing response, suggests that this antigen more effectively elicits affinity maturation as an outcome of the GC response than Hla_{H}. The GC response necessitates a dynamic interaction between antigen-presenting cells, B cells, and T cells within the draining lymph node (dLN). While antigen-specific T cell activation is required to initiate the dLN response to vaccination, differentiated T follicular helper (T_{FH}) cells are essential for affinity-based positive selection of GC B cell. CXCR5 upregulation in a subset of activated T cells within the interfollicular zone of the dLN enables T cell localization to the nascent GC and upregulation of Bcl6, the lineage-defining transcription factor of T_{FH} cells. The essential role of the T_{FH} compartment in B cell maturation in the GC is evident in Bcl6 knockout mice which are devoid of GCs and thereby unable to generate a mature antibody response. To evaluate whether the nature of the GC response distinguishes the Hla_{H} and Hla_{HRE} antigens, the kinetics of the developing anti-Hla neutralizing response to vaccination were studied. Owing to blood volume and tissue limitations in neonatal mice, five week-old mice were subjected to prime-boost vaccine delivery with each antigen adjuvanted with Alum. As early as 1 week post-boost, a significant increase in the Hla-neutralizing antibody response was elicited by Hla_{HRE} relative to that of Hla_{H} (FIG. 6A). By 3 weeks post-boost when the elicited antibody response is expected to be fully matured, an increase in the neutralizing antibody titer was seen for both Hla_{H} and Hla_{HRE} antigens, however the HlaH-elicited response remained lower in magnitude than that of the Hla_{HRE}-elicited response (FIG. 6A). The functional attributes of these elicited antibodies were evaluated by assessing blockade of radiolabeled Hla binding (FIG. 6B) and oligomerization (FIG. 6C) on rabbit red cells. In both studies, Hla_{HRE}-elicited antibodies exhibited enhanced performance compared to those elicited by HlaH.

These studies were extended to directly evaluate the cellular response within the vaccine-draining iliac lymph node response at day 7 following prime-boost vaccination with either Hla_{H} or Hla_{HRE}. No differences was observed in either the total population of CD19+GC B cells marked by co-expression of GL7/Fas (FIG. 6D) or the proliferating (Ki67+) CD19+GL7⁺Fas⁺B cells (FIG. 6E). Similarly, no significant difference was observed in the dLN total CD4⁺ T cell compartment between the two vaccines (FIG. 6F). In contrast, an increase in median CXCR5 fluorescence intensity was observed in the bulk CD4+population (FIG. 6G) and Ki67+CD4+cells (FIG. 6H) in Hla_{HRE}-immunized mice. As CXCR5 expression is required for early T_{FH} differentiation to render these cells capable of migration to the nascent germinal center, this finding suggests that the Hla_{HRE} antigen amplifies the pre-T_{FH} response.

It was hypothesized that Hla_{HRE} vaccine-mediated protection may elicit amplification of the T_{FH} response to infection. The CXCR5+compartment in the dLN of vaccinated mice was examined, 7 days following secondary S. aureus SSTI. Following infection, clinical lesions remained prominent in the sham and Hla_{H} vaccinated mice, however were limited in the Hla_{HRE} vaccine recipients (FIG. 6I). As the T_{FH} population is relatively rare in the dLN, we first utilized mass cytometry to analyze pooled dLN to gate on CXCR5+cells that co-express ICOS and PD1, revealing an increase in the CD4+CXCR5⁺ICOS⁺PD1⁺ compartment in Hla_{HRE} vaccinated mice relative to that in Hla_{H} vaccinated mice (FIG. 6L). To enhance the specificity of this analysis, we utilized a flow cytometric approach to detect CXCR5hi CD4⁺ T cells that co-express Bcl6 in individual dLN harvested following S. aureus infection. Compared to sham and Hla_{H} vaccinated mice, animals receiving Hla_{HRE} exhibited expansion of the CD4+CXCR5hi Bc16+T_{FH} population (FIG. 6J).

Next, it was examined if the beneficial effects of Hla_{HRE} vaccination reflected the role of Hla in modulation of the T cell compartment. While prior studies have revealed that Hla blunted the antigen-specific T cell response, these studies do not encompass evaluation of the functional specificity of the response. Groups of mice were infected with wild-type S. aureus or its isogenic Dhla variant and dLN subjected to mass cytometry 7 days post-primary infection. Deletion of Hla was associated with an increase in the CXCR⁵⁺ICOS⁺PD1⁺ T_{FH} population (FIG. 6M). Analysis of individual mice infected with WT S. aureus, Dhla, and the Dhla variant complemented with a plasmid that enables restoration of Hla expression (Dhla::phla) confirmed that Hla expression negatively impacts the CXCR5^{hi}Bc16⁺T_{FH} population (FIG. 6K).

There is an increasing appreciation in the S. aureus field that vaccine failures may in part result from vaccine delivery to a non-naive host that harbors a pre-existing immune response that becomes amplified by vaccination. The protective efficacy of the Hla_{HRE} vaccine was assessed in two clinical contexts in which pre-existing immunity is relevant: passive transfer of maternal antibodies that may modulate vaccine antigen bioavailability and/or antigenicity, and in the setting of prior S. aureus infection. First, studies were conducted to test whether Hla_{HRE}-immunized dams conferred protection against S. aureus infection to their offspring (FIG. 7A). Pups from Hla_{HRE}-immunized dams exhibited protection against skin abscess and dermonecrosis (FIG. 7B, blue) compared to offspring of Alum-immunized dams (black), reflecting the neutralizing anti-Hla response (FIG. 7C). To evaluate whether passive transfer of maternal antibodies impaired the protective response to subsequent infant vaccination, it was examined whether circulating anti-Hla antibodies in offspring adversely influenced the clinical outcome of active vaccination. Pups from sham (gray) and Hla_{HRE}-immunized (green) dams received Hla_{HRE} vaccination followed by S. aureus infection. Both groups manifest protection against S. aureus infection as observed in pups born to mice that received passive immunity from maternal immunization (blue), suggesting that prior maternal immunity does not negatively influence the functional outcome of active immunization. These maternal-infant studies were extended by evaluation of the duration of the serologic response in offspring, recognizing that passively transferred antibodies are reported to exist in murine offspring up to ˜15 weeks of life. Mice from each of the vaccine groups were sampled at 6, 8, 10, and 16 weeks of life and assessed for serum Hla neutralizing properties. A robust passive transfer of anti-Hla neutralizing function was observed, consistent with the observed protection following maternal immunization (FIG. 7G blue). Active immunization of pups following maternal Hla_{HRE} immunization (green) was indistinguishable from that observed in Hla_{HRE}-vaccinated pups born to dams that received sham vaccination with alum alone (gray), signifying that exposure to maternal anti-Hla antibody does not incur functional loss of the Hla_{HRE} vaccine response in offspring. A progressive decrease in the neutralizing antibody titer occurred in all groups over 10-16 weeks as expected based on known kinetics of the vaccine response. Upon re-infection of each group of mice with S. aureus, we observed a recall anti-Hla serologic response in mice that had received active immunization with Hla_{HRE} either in the absence or presence of maternal Hla_{HRE} vaccine.

To extend these studies toward direct evaluation of whether prior S. aureus infection modulated the protective efficacy of Hla_{HRE} vaccination, groups of naive mice were subjected to S. aureus SSTI at 5 weeks of age followed by a prime-boost vaccine regimen with alum alone or alum-adjuvanted Hla_{HRE} vaccines (FIG. 7D). Hla_{HRE} conferred near complete protection against infection on the contralateral flank two weeks following the boost (FIG. 7E), reflecting the neutralizing anti-Hla response (FIG. 7F). Together, these findings suggest that vaccination with the Hla_{HRE} antigenic variant may not be as susceptible to the deleterious impact of pre-existing immunity on vaccine outcomes.

To understand the versatility of the Hla_{HRE} vaccine, protective efficacy was assessed in a series of distinct biological settings. S. aureus skin infection is commonly modeled in male mice owing to both the epidermal structure of the skin which facilitates histopathologic analysis and magnitude of the lesional differences that result from male hormone-mediated enhancement in S. aureus virulence. First, the Hla_{HRE} vaccine efficacy in protecting female mice against severe SSTI following neonatal vaccination was demonstrated (FIG. 8A), consistent with elicitation of the anti-Hla neutralizing antibody response (FIG. 8B). Second, the protective efficacy in young mice that were not exposed to vaccination as neonates was evaluated. Groups of 3 week-old naive mice received a priming immunization with either alum alone or alum-adjuvanted Hla_{HRE} followed by a boost 14 days later (FIG. 8C). When subjected to primary S. aureus infection, Hla_{HRE}-vaccinated mice exhibited protection from infection (FIG. 8D), also reflecting a productive neutralizing anti-Hla antibody response (FIG. 8E).

Successful vaccine design, evaluation, and implementation in the human population requires both antigen optimization and knowledge of the mechanism by which distinct adjuvants amplify desired host immune responses. It was first ensured that a non-tagged variant of the Hla_{HRE} antigen was functional. Neonatal vaccination with both the tagged Hla_{HRE} (HIS-Hla_{HRE}) and the untagged moiety elicited significant protection against S. aureus skin infection (FIG. 9A). While a minor improvement in the clinical efficacy of the untagged variant was observed early in the course of infection, the neutralizing IC₅₀ was nearly identical for both antigens (FIG. 9B). The observed adjuvant-specific differences (Alum, AddaSO3, MPLA+alum, and CpG-ODN1585) in potency of Hla_{HRE} vaccine-mediated protection as provided in Example 4 reflected the magnitude of the T_{FH} response in the dLN. Evaluating the relationship of T_{FH} cell recovery and clinical abscess size, an inverse correlation was observed (FIG. 9C). In accord with this finding, a direct correlation between the IC₅₀ and T_{FH} response was observed (FIG. 9D). Together, these findings underscore the importance of the T_{FH} response as an immune correlate of protective efficacy of the Hla_{HRE} vaccine, and suggest that the magnitude of this response can be modulated by vaccine formulation.

### Example 6: Discussion

These findings leverage the observation that the anti-Hla response is a defined correlate of human protective immunity to S. aureus in the pediatric population. Through population-level analysis characterizing the development anti-Hla response in childhood, a specific window of opportunity was defined in the first two years of life to augment this functional response. A novel candidate Hla antigen, Hla_{HRE}, distinguished by its ability to elicit a robust toxin-neutralizing response when delivered in the context of pre-clinical vaccination of neonatal mice was developed. An analysis of the mechanism of protective immunity elicited by Hla_{HRE} revealed the importance of the T_{FH} as a functional correlate of Hla_{HRE} vaccine-mediated protection—suggest that prior exposure to Hla (which is nearly ubiquitous in adults based on serologic findings) is not an a priori limitation to successful elicitation of a protective immune response to Hla_{HRE} vaccination.

It has been recognized that vaccine development to target S. aureus has suffered from a lack of understanding of the T cell response to this pathogen. Consequently, vaccines have not been designed to elicit T cell specific correlates of immune protection. Hla neutralization is essential for protection of the antigen-specific T cell response during infection, thus providing mechanistic insight on the observed clinical efficacy of the Hla_{HRE} vaccine. Detoxified Hla variants (including HlaH35L, HlaH35L/H48A) have previously been considered or are being evaluated for vaccine development. The studies disclosed herein illustrate that the specific antigenic form of Hla influences the magnitude of the neutralizing antibody response, revealing that some Hla variants may only generate modest vaccine protection. While it is possible that this response difference may be overcome by delivery of higher quantities of antigen or alternatively through booster regimens, the potency of the Hla_{HRE} antigen in amplification of the host immune response may be advantageous to enable antigen sparing and in settings such as the neonate and the elderly where the vaccine-elicited response may be limited by inherent attributes of host immunity.

At present, the precise molecular mechanism by which the Hla_{HRE} antigen amplifies the T_{FH} response and affinity maturation of anti-Hla antibodies as a consequence of initial vaccine delivery is not fully understood. Antigen specificity and the duration of antigen availability to GC B cells are primary determinants of the GC response. The observed increase in CXCR5 expression on CD4⁺ T cells following delivery of Hla_{HRE}. compared with that observed upon Hla_{H} immunization suggests that Hla_{HRE} more effectively stimulates an IL-21 response in the dLN, as this cytokine is essential for T_{FH} differentiation. It was highly unexpected that two amino acid substitutions in Hla_{HRE} relative to Hla_{H} would markedly alter the outcome of vaccination. A detailed analysis of antigen trafficking kinetics, cellular uptake, duration of antigen availability in the GC will be required to fully elucidate the biological differences that underlie antigen specificity observed. As Hla_{H} and Hla_{HRE} are functionally distinguished by ADAM10 binding capability, it will be of interest to understand whether cellular trafficking of the Hla-ADAM10 complex on both professional and non-professional antigen presenting cells modulates antigen distribution and processing.

As the unique attributes of Hla_{HRE} as an antigen are observed upon formulation with multiple distinct adjuvants, the disclosed findings provide several opportunities to enhance the development of a S. aureus vaccine. First, they compel a focus on the vaccine antigen itself to optimize vaccination outcome. Whether Hla_{HRE} is considered for use in a monovalent context or in the presence of other vaccine antigens, its biological properties are expected to augment the elicited Hla neutralizing antibody response. Second, these studies indicate that the T_{FH} response may serve as a second immunologic correlate of vaccine-mediated protection that can be evaluated and potentially targeted in human populations through choice of adjuvant, which may vary dependent on the vaccine-targeted population. Finally, these studies further underscore the importance of Hla neutralization by a S. aureus vaccine. As a toxin that interferes with the development of T cell-mediated immunity to S. aureus, Hla neutralization is expected to not only protect against Hla-mediated cellular injury in disease but perhaps more importantly, ensure that the T cell compartment remains viable and poised to support the generation of a diverse immune response to the pathogen. Delivery of an Hla_{HRE}-containing vaccine early in life may therefore enable protection against S. aureus infection early in life, but simultaneously permit the success of S. aureus vaccines developed for administration later in life to protect against disease-specific virulence factors. The Hla_{HRE} vaccine may thus provide a novel opportunity to advance development of a population-level S. aureus vaccine.

### Example 7: Methods

### Participant Population and Sample and Data Collection

This study was approved by the University of Chicago Institutional Review Board. Written informed consent was obtained for all participants. Participants were children 18 years and younger undergoing sedated procedures or imaging studies at Comer Children's Hospital. Upon enrollment, study personnel collected swab samples from the anterior nares, axillae, and inguinal folds of each participant to determine S. aureus colonization status via culture-based methods. Participants or their parents/legal guardians were asked to complete a survey regarding their demographics, current and past medical history, and history of S. aureus infection or known skin and soft tissue infection in the participant or a household member. Lastly, serum samples were obtained from each participant. Samples were de-identified and stored in a secure repository at −80° C.

### ELISA and Antibody Neutralization Assays

Human serum analysis. NUNC MAXISORP™ 384 well plates (Fisher Scientific) were coated with Hla_{H35L} at 1 μg/ml in PBS and incubated overnight at 4° C. Plates were blocked with 0.1% bovine serum albumin (BSA) in PBS for 2 hours at room temperature. Patient sera samples were diluted 1:10 and then two-fold for a total of 24 dilutions and transferred to the 384 MAXISORP™ plates for 1 hour at room temperature. Plates were washed three times with PBS/0.05% Tween-20 and incubated with goat anti-human HRP conjugated antibody (Southern Biotech) at a 1:20,000 dilution for 1 hour at room temperature. Plates were washed three times and developed for 30 minutes at room temperature with QUANTABLU™ Fluorogenic Peroxidase Substrate Kit and reactions were stopped with QUANTABLU™ Stop Solution. Fluorometric detection was measured using a TECAN INFINITE M200 PRO plate reader at excitation 320 nm and emission 400 nm. For the neutralization assay, patient sera samples were diluted 1:10 in 0.1% BSA/PBS and incubated with purified toxin for 1 hour at room temperature. After incubation, 5×10⁷ of prewashed rRBCs were added and incubated for 1 hour at room temperature on a platform shaker. The final concentration of toxin for the assays was 2 nM. Cells were pelleted and supernatant absorbance readings (450 nm) were used to generate non-linear regression curves with log (inhibitor) vs. response—variable slope curves using GraphPad Prism software.

Juvenile murine serum analysis. Sera was diluted 1:25 and then by four-fold serial dilutions for eight dilutions total. Anti-Hla titers were determined by ELISA as previously described and ELISA absorbance readings (450 nm) used for generation of four-parameter log(dose)-response curves using Prism software. For the neutralization assay, serum was diluted 1:100 and then two-fold for eight dilutions total. The diluted serum was incubated with 2 nM purified toxin for 15 minutes at room temperature before 5×10⁷ rRBCs were added then incubated at room temperature for 1 hour. Cells and debris were pelleted and supernatant absorbance readings (450 nm, TECAN INFINITE M200 Pro) were used to generate non-linear regression curves with log (inhibitor) vs. response—variable slope curves using GraphPad Prism software. Both the ELISA and neutralization assays were performed in triplicate.

Neonatal murine serum analysis. 384-well MAXISORP™ plates (Thermo Fisher) were coated with 50 μL of 1 μg/mL Hla_{H35L} and incubated overnight at 4° C. Plates were blocked with 0.1% BSA in PBS for 2 hours at room temperature, following incubation with 15 μL of neonatal pre-infection sera for 1 hour at room temperature. Plates were washed three times with PBS/0.05% Tween-20 and then incubated with 15 μL of goat anti-mouse IgG-HRP antibody (Southern Biotech) at a 1:20,000 dilution for 1 hour at room temperature. Plates were washed three times and developed for 15 minutes at room temperature with 20 μL of TMB substrate kit (Thermo Scientific Pierce P134021) and the reaction stopped with 20 μL of 4N sulfuric acid (Fisher Scientific). Absorbance values were read at 450 nm using a Tecan Infinite M200 Pro plate reader.

For neutralization assays, neonatal pre-infection sera was diluted two-fold for a total of 16 dilutions and incubated for 1 hour at room temperature with 0.8 pM purified Hla. After incubation, 10 μL of the toxin/sera mixture was added to 1×10⁷ of prewashed rabbit red cells and incubated for 1 hour at room temperature in a v-bottom 384 well plate (Thomas Scientific). Cells and debris were pelleted and supernatant absorbance readings (450 nm, Tecan Infinite M200 Pro) were used to generate non-linear regression curves with log (inhibitor) vs. response—variable slope curves using GraphPad Prism software.

Adult murine serum analysis with high-dose immunization. Sera was collected 1 week, 2 weeks and 3 weeks post prime and boost 20 pg vaccinations. Sera was diluted 1:4 in PBS and assays were performed as described above.

### Plasmid Construction and Hla Antigen Purification

Toxin variants were cloned into the pET24b expression vector containing a C-terminal polyhistidine tag. Hla_{H35L} was previously generated as described 5. Hla_{D45A/Y118F} was generated via site-directed mutagenesis with template DNA from a pET24b construct containing wildtype Hla cDNA previously generated. The following oligos were utilized: D45A sense-5′ GTATTTTATAGTTTTATCGATGCTAAAAATCACAATAAAA 3′ (SEQ ID NO: 39); D45A antisense 5′ TTTTATTGTGATTTTTAGCATCGATAAAACTATAAAATAC 3′(SEQ ID NO: 40); Y1 18F sense-5′ GTATATGAGTACTTTAACTTTTGGATTCAACGGTAATGTTA 3′ (SEQ ID NO: 41); Y118F antisense-5′ TAACATTACCGTTGAATCCAAAAGTTAAAGTACTCATATAC 3′(SEQ ID NO: 42). Hla_{R66C/E70C} was previously generated in the Bubeck Wardenburg lab as a GST fusion protein in pGEX6P1. To generate a polyhisitidine tagged protein, template DNA from the pGEX6P1 construct containing R66C/E70C cDNA was used with primers for restriction site cloning using Xbal and Xhol (New England Biolabs) into pET24b vector.

The following primers were used: sense-5′ CGGCGGCTCGAGATTTGTCATTTCTTCTTT 3′; (SEQ ID NO: 43), antisense-5′ CGGCGGTCTAGAAGGAGGATATATATGGCAGATTCTGATATAATATT 3′ (SEQ ID NO: 44). Site-directed mutagenesis was used to incorporate the H35L mutation in Hla_{D45/Y118F} and Hla_{R66C/E70C} constructs with the following primers: sense-5′ CTTATGATAAAGAAAATGGCATGCTCAAAAAAGTATTTTATAGTTTTATCGATG 3′ (SEQ ID NO: 45); antisense-5 ′CATCGATAAAACTATAAAATACTTTTTTGAGCATGCCATTTTCTTTATCATAAG 3′ (SEQ ID NO: 46). Mutagenesis reactions were Dpnl (New England Biolabs) digested and transformed into Escherichia coli DH5a on selection agar. Each construct was sequenced (Azenta Life Sciences) for verification. Confirmed clones were then transformed into E. coli BL21 for recombinant protein expression and purification.

Peptide sequences Hla₅₀, Hla_{P1}, and Hla_{P2} constructs were generated by SynBio Technologies, and constructs transformed into BL21 for protein expression and purification. Recombinant toxin variants were purified using standard protocols described previously, LPS extracted and evaluated by 10% SDS-PAGE followed by Coomassie blue staining.

### Characterization Assays for Hla Antigens

Hemolysis assay. Rabbit red blood cell (rRBC) hemolysis was assayed by incubation of rRBCs (Hemostat Labs) with purified toxin variants ranging from 0.08-10 μg/mL for 1 hour at room temperature. Following incubation, cells were pelleted by centrifugation and supernatant absorbance at 450 nm measured using a Tecan Infinite M200 Pro plate reader. Percent rRBC hemolysis were calculated relative to 1% Triton X-100 maximal lysis controls. The assays were performed in triplicate and repeated for reproducibility on separate days.

Radiolabeled toxin binding and oligomerization assay. Hla was synthesized by in vitro transcription and translation in an E. coli S30 extract (Promega) supplemented with T7 RNA polymerase, rifampin (rifampicin), and [³⁵S]methionine according to the manufacturer's instructions. For the binding assay, 125 μL of 12.5% rabbit red blood cells (rRBC) in K-PBSA/3ME (20 mM potassium phosphate [monobasic], 150 mM NaCl pH 7.4, 1 mg/ml bovine serum albumin, 1 mM β-mercaptoethanol) was incubated with 10 μl of radiolabeled Hla mixture for 5 minutes at room temperature. Cells were pelleted, washed twice with ice-cold PBS, resuspended in 200 μl of PBS and added to scintillation fluid (Research Products International, Econo-Safe). Radioactivity from cell bound toxin was quantified on a Beckman LS6000 scintillation counter. For oligomerization assays, rRBCs were used as described above but incubated with 10 μl of the radiolabeled Hla mixture (˜1 nM) for 1 h at room temperature. Following incubation, cells were pelleted and washed with 500 μl K-PBSA/3ME and then resuspended in 90 μl 1x Laemmli buffer. Samples were divided into 30 μl aliquots and incubated at 37° C., 62° C. and 80° C. for 10 min before samples were loaded onto 10% sodium dodecyl sulfate (SDS)-PAGE gels for electrophoresis. The gels were dried, and then the results were visualized using a phosphorimager (GE Healthcare Typhoon Trio Imager). To analyze Hla_{HRE} oligomerization capability, rRBCs were treated as described above with 10 μl of increasing concentration of radiolabeled Hla mixture (1x=˜1 nM; 2x=˜2 nM and 5x=˜5 nM) and incubated at 37° C. before samples were separated by SDS-PAGE and prepared as above. For binding and oligomerization assays using vaccinated mouse sera, each sera sample was diluted 1:5 in PBS, then incubated with 10 μl of radiolabeled Hla for 10 mins at room temperature before adding the rabbit red cells. Assays were performed as described above. Binding assays were performed in triplicate and repeated for reproducibility on separate days. Oligomerization assays were performed with a single replicate and repeated for reproducibility on separate days.

Proteomics disulfide bond analysis of H/a_{HRE}. Purified protein sample was digested with 250 ng trypsin for overnight incubation at 37° C. The resulting peptides were desalted using the C18 column, and the eluates were dried under a SpeedVac vacuum concentrator. Peptides were analyzed by LC-MS/MS using a Vanquish Neo UHPLC System coupled to an ORBITRAP ECLIPSE™ Tribrid Mass Spectrometer with FAIMS Pro Duo interface (Thermo Fisher Scientific). The sample was loaded on a Neo trap cartridge coupled with an analytical column (75 μm ID×50 cm PEPMAP™ Neo C18, 2 μm). Samples were separated using a linear gradient of solvent A (0.1% formic acid in water) and solvent B (0.1% formic acid in ACN) over 120 minutes. For MS acquisition, FAIMS switched between CVs of −35 V and −65 V with cycle times of 1.5 s per CV. MS1 spectra were acquired at 60,000 resolution with a scan range from 380 to 1400 m/z, normalized AGC target set at Standard mode, and maximum injection time set at Auto mode. Precursors were filtered using monoisotopic peak determination set to peptide, charge state 3 to 8, dynamic exclusion of 60 s with ±10 ppm tolerance. For the MS2 analysis, the isolated ions were fragmented by assisted higher-energy collisional dissociation (HCD) at 25% and 30% and acquired in an orbitrap at 30,000 resolution. The AGC and maximum IT were 200% and 70 ms, respectively.

The acquired MS/MS data was queried for disulfide cross-link identification against a target protein sequence using MeroX 2.0 software. The Disulfide bond was selected as the cross-linker, and the maximum number of missed cleavages was set to 4. The precursor mass tolerance was set to 20 ppm, and the fragment mass tolerance was set to 50 ppm. The search results were validated with 5% FDR and precise scoring setting.

Thermal Shift Assay. The stability of each toxin variant was measured by quantifying the melting temperature of each toxin variant (50% denaturation point) compared to the wild-type toxin. Each well contained a final concentration of 5pM protein and 1:20 dilution of 5x SYPRO™ Orange stock (Thermo Fisher S6650) in 20 mM Tris pH 7.5 and 150 mM NaCl buffer. The plates were heated in STEPONEPLUS™ Real-Time PCR System (Thermo Fisher) from 20 to 90° C. in increments of 0.3° C. The wavelengths for excitation and emission were 490 and 575 nm, respectively.

### Bacterial Strains

S. aureus strains USA300/LAC and the isogenic USA300/LAC Δhla mutant were grown in tryptic soy broth (Bacto TSB, Fisher Scientific) overnight at 37° C. with agitation for 14-16 hours. The strains were subcultured 1:100 in TSB the following day and prepared as previously described for infection modeling.

### Animal Modeling

Vaccine Preparation and Immunization. Vaccine antigens were LPS extracted and prepared with ALHYDROGEL®(InvivoGen), 1:1 antigen to AddaSO3 (InvivoGen, 10253-42-02), AS04-like formulation with antigen plus 500 g/mL ALHYDROGEL®(InvivoGen, vac-alu-250) and 50 g/mL of MPLA-SM VacciGrade (InvivoGen, vac-mpla), or CpG-ODN1585 (InvivoGen) formulation with antigen plus 2pg of CpG-ODN1585. For immunization of juvenile and adult mice, 20 micrograms of each antigen was delivered via intramuscular (IM) injection to cohorts of 3-week-old C57BL/6J male or female mice on days 0 and 14. For immunization of neonatal mice, 2 g of each antigen was delivered in a total volume of 10 I IM to litters of C57BL/6 pups between P1-P3 with a boost delivered on P21 (day of weaning).

Skin and Soft Tissue Infection Modeling. All animal experiments were reviewed, approved, and supervised by the Institutional Animal Care and Use Committee (IACUC) at Washington University in St. Louis. To determine antibody titers, serum was collected from 5 mice per group, one week after boost immunizations and prior to S. aureus challenge. C57BL/6 male mice 5-6 weeks of age were anesthetized via intraperitoneal injection of ketamine (20 mg/kg) and xylazine (5 mg/kg) followed by right flank subcutaneous challenge with 1×10⁸ CFU S. aureus strain USA300/LAC in 50 μL PBS. Lesional abscess and dermonecrosis area (mm2) was measured at 24-hour intervals for 10 days. Mice received secondary infection on the left flank, 21-28 days after the start of the primary infection. Abscess and dermonecrosis size for all studies was determined according to the formula A=(π/2)(length mm)(width mm).

### Cell-Based Immunologic Assays

Mass cytometry analysis. Ten days following either primary or secondary infection as indicated, the inguinal draining lymph node (dLN) was harvested from the ipsilateral side of S. aureus infected mice and processed to obtain single cell suspensions. A minimum of three dLN from independent mice were pooled together for each condition. Cells (3×10⁶ per sample) were subjected to cisplatin labelling followed by staining for cell surface markers and intracellular antigens of interest with the antibody reagents as noted in Table 2.

TABLE 2
Antibody Reagents Utilized for Mass Cytometry

| Label | Target | Clone | Manufacturer | Cat Number |
|---|---|---|---|---|
| 089Y | CD45 | 30-F11 | Fluidigm | 3089005B |
| 142Nd | CXCR5/CD185 | 614641 | Fluidigm | 3142008B |
| 145Nd | CD69 | H1.2F3 | Fluidigm | 3145005B |
| 146Nd | CD8a | 53-6.7 | Fluidigm | 3146003B |
| 148Nd | CD278/ICOS | C398.4A | Fluidigm | 3148019B |
| 149Sm | CD19 | 6D5 | Fluidigm | 3149002B |
| 150Nd | CD44 | IM7 | Fluidigm | 3150018B |
| 151Eu | CD25 | 3C7 | Fluidigm | 3151007B |
| 152Sm | CD3 | 145-2C11 | Fluidigm | 3152004B |
| 158Gd | FoxP3 | FJK-16s | Fluidigm | 3158003A |
| 159Tb | CD184/CXCR4 | L276F12 | Fluidigm | 3159030B |
| 161Dy | Ki67 | B56 | Fluidigm | 3161007B |
| 164Dy | CD62L | MEL-14 | Fluidigm | 3164003B |
| 172Yb | CD4 | RM4-5 | Fluidigm | 3145002B |
| 175Lu | PD-1 | RMP1-30 | Thermo | 14-9981-85_B |

Fixation and cell permeabilization for intracellular staining were performed with the EBIOSCIENCE™ Permeabilization Buffer and FoxP3 Transcription Factor Staining Buffer Set according to the manufacturer's protocol. Stained cells were washed, barcoded by sample, pooled, and analyzed on a Fluidigm CyTOF2 HELlOS™ mass cytometer by the Bursky Center for Human Immunology and Immunotherapy Programs at Washington University. De-barcoded samples were analyzed using Cytobank software (Beckman Coulter).

### Statistical Analyses

Statistical analysis for serologic studies and all in-vitro assays was performed with GraphPad Prism software using one-way ANOVA with Tukey's or Dunn's multiple comparison test or independent T-test, p<0.05. Assessment of statistical significance in skin infection studies was performed with GraphPad Prism software using 2-way ANOVA with Tukey's multiple comparison test, p<0.05.

List of sequences

| SEQ | | |
|---|---|---|
| ID | | |
| NO | TYPE | SEQUENCE |
| 1 | WT | ADSDINIKTGTTDIGSNTTVKTGDLVTYDKENGMHKKVFYSFIDDKNHN |
| | | KKLLVIRTKGTIAGQYRVYSEEGANKSGLAWPSAFKVQLQLPDNEVAQI |
| | | SDYYPRNSIDTKEYMSTLTYGFNGNVTGDDTGKIGGLIGANVSIGHTLK |
| | | YVQPDFKTILESPTDKKVGWKVIFNNMVNQNWGPYDRDSWNPVYGNQ |
| | | LFMKTRNGSMKAADNFLDPNKASSLLSSGFSPDFATVITMDRKASKQQ |
| | | TNIDVIYERVRDDYQLHWTSTNWKGTNTKDKWTDRSSERYKIDWEKEE |
| | | MTN |
| 2 | WT-DNA | ATCGATTACATTTTTAAATCAAACAAATCATTAGTTTTAAAGAAATTAA |
| | | TCAATAGAATTAGCTATGTCTTTTCCTTGTTTCATAAAGAATTTATTCA |
| | | ACTTTGACTAACCCTCGAAATTGAAATGCTTCCTTTCAAATTTTAAAT |
| | | AAAAGTTAAAAACATATTCTTAAAATAATCACTCATCATCACTCAGTA |
| | | ATTTATCAGTTGCTATATTAAATATACTAATATAAACGATTATAAATAT |
| | | TTGATATGTCTCAACTGCAAATATTCTAAATTGACATATTGATTATTGT |
| | | TTCCTCAAATCGTTCAAAAAAATAGAAGGATGATGAAAATGAAAACA |
| | | CGTATAGTCAGCTCAGTAACAACAACACTATTGCTAGGTTCCATATT |
| | | GATGAATCCTGTCGCTGGTGCCGCAGATTCTGATATTAATATTAAAA |
| | | CCGGTACTACAGATATTGGAAGCAATACTACAGTAAAAACAGGTGAT |
| | | TTAGTCACTTATGATAAAGAAAATGGCATGCACAAAAAAGTATTTTAT |
| | | AGTTTTATCGATGATAAAAATCACAATAAAAAACTGCTAGTTATTAGA |
| | | ACGAAAGGTACCATTGCTGGTCAATATAGAGTTTATAGCGAAGAAGG |
| | | TGCTAACAAAAGTGGTTTAGCCTGGCCTTCAGCCTTTAAGGTACAGT |
| | | TGCAACTACCTGATAATGAAGTAGCTCAAATATCTGATTACTATCCAA |
| | | GAAATTCGATTGATACAAAAGAGTATATGAGTACTTTAACTTATGGAT |
| | | TCAACGGTAATGTTACTGGTGATGATACAGGAAAAATTGGCGGCCTT |
| | | ATTGGTGCAAATGTTTCGATTGGTCATACACTGAAATATGTTCAACCT |
| | | GATTTCAAAACAATTTTAGAGAGCCCAACTGATAAAAAAGTAGGCTG |
| | | GAAAGTGATATTTAACAATATGGTGAATCAAAATTGGGGACCATATG |
| | | ATAGAGATTCTTGGAACCCGGTATATGGCAATCAACTTTTCATGAAA |
| | | ACTAGAAATGGTTCTATGAAAGCAGCAGATAACTTCCTTGATCCTAA |
| | | CAAAGCAAGTTCTCTATTATCTTCAGGGTTTTCACCAGACTTCGCTA |
| | | CAGTTATTACTATGGATAGAAAAGCATCCAAACAACAAACAAATATA |
| | | GATGTAATATACGAACGAGTTCGTGATGATTACCAATTGCATTGGAC |
| | | TTCAACAAATTGGAAAGGTACCAATACTAAAGATAAATGGACAGATC |
| | | GTTCTTCAGAAAGATATAAAATCGATTGGGAAAAAGAAGAAATGACA |
| | | AATTAATGTAAATTATTTGTACATGTACAAATAAATATAATTTATAACT |
| | | TTAGCCGATAACTTCAGAAATGATGTTTTTCGGCTAATTTTTATACTT |
| | | AATTTAGTTTAATTAACTTATGATATTTTTAATTTAAACTATGTACTTG |
| | | ATTTGCTTTCCTGACTTAAAAATTAGAGCTAAACATGACACTATAGAG |
| | | GTGTTTT |
| 3 | H35X, X | ADSDINIKTGTTDIGSNTTVKTGDLVTYDKENGMXKKVFYSFIDDKNHNK |
| | is any aa | KLLVIRTKGTIAGQYRVYSEEGANKSGLAWPSAFKVQLQLPDNEVAQIS |
| | | DYYPRNSIDTKEYMSTLTYGFNGNVTGDDTGKIGGLIGANVSIGHTLKY |
| | | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 4 | | KKVFYSFIDDKNHNK |
| 5 | | GPGPG |
| 6 | H35X, | ADSDINIKTGTTDIGSNTTVKTGDLVTYDKENGMXKKVFYSFIDDKNHNK |
| | R66C | KLLVIRTKGTIAGQYCVYSEEGANKSGLAWPSAFKVQLQLPDNEVAQIS |
| | | DYYPRNSIDTKEYMSTLTYGFNGNVTGDDTGKIGGLIGANVSIGHTLKY |
| | | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 7 | H35L, | ADSDINIKTGTTDIGSNTTVKTGDLVTYDKENGMLKKVFYSFIDDKNHNK |
| | R66C | KLLVIRTKGTIAGQYCVYSEEGANKSGLAWPSAFKVQLQLPDNEVAQIS |
| | | DYYPRNSIDTKEYMSTLTYGFNGNVTGDDTGKIGGLIGANVSIGHTLKY |
| | | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 8 | H35X | ADSDINIKTGTTDIGSNTTVKTGDLVTYDKENGMXKKVFYSFIDDKNHNK |
| | E70C | KLLVIRTKGTIAGQYRVYSCEGANKSGLAWPSAFKVQLQLPDNEVAQIS |
| | | DYYPRNSIDTKEYMSTLTYGFNGNVTGDDTGKIGGLIGANVSIGHTLKY |
| | | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 9 | H35L | ADSDINIKTGTTDIGSNTTVKTGDLVTYDKENGMLKKVFYSFIDDKNHNK |
| | E70C | KLLVIRTKGTIAGQYRVYSCEGANKSGLAWPSAFKVQLQLPDNEVAQIS |
| | | DYYPRNSIDTKEYMSTLTYGFNGNVTGDDTGKIGGLIGANVSIGHTLKY |
| | | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 10 | H35X | ADSDINIKTGTTDIGSNTTVKTGDLVTYDKENGMXKKVFYSFIDDKNHNK |
| | R66C | KLLVIRTKGTIAGQYCVYSCEGANKSGLAWPSAFKVQLQLPDNEVAQIS |
| | E70C | DYYPRNSIDTKEYMSTLTYGFNGNVTGDDTGKIGGLIGANVSIGHTLKY |
| | | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 11 | H35L | ADSDINIKTGTTDIGSNTTVKTGDLVTYDKENGMLKKVFYSFIDDKNHNK |
| | R66C | KLLVIRTKGTIAGQYCVYSCEGANKSGLAWPSAFKVQLQLPDNEVAQIS |
| | E70C | DYYPRNSIDTKEYMSTLTYGFNGNVTGDDTGKIGGLIGANVSIGHTLKY |
| | | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 12 | I5A, I7A, | ADSDANAKTGTTDIGSNTTVKTGDLVTYDKENGMLKKVFYSFIDDKNH |
| | H35L | NKKLLVIRTKGTIAGQYCVYSEEGANKSGLAWPSAFKVQLQLPDNEVA |
| | R66C | QISDYYPRNSIDTKEYMSTLTYGFNGNVTGDDTGKIGGLIGANVSIGHTL |
| | | KYVQPDFKTILESPTDKKVGWKVIFNNMVNQNWGPYDRDSWNPVYGN |
| | | QLFMKTRNGSMKAADNFLDPNKASSLLSSGFSPDFATVITMDRKASKQ |
| | | QTNIDVIYERVRDDYQLHWTSTNWKGTNTKDKWTDRSSERYKIDWEK |
| | | EEMTN |
| 13 | I5A, I7A, | ADSDANAKTGTTDIGSNTTVKTGDLVTYDKENGMLKKVFYSFIDDKNH |
| | H35L | NKKLLVIRTKGTIAGQYRVYSCEGANKSGLAWPSAFKVQLQLPDNEVA |
| | E70C | QISDYYPRNSIDTKEYMSTLTYGFNGNVTGDDTGKIGGLIGANVSIGHTL |
| | | KYVQPDFKTILESPTDKKVGWKVIFNNMVNQNWGPYDRDSWNPVYGN |
| | | QLFMKTRNGSMKAADNFLDPNKASSLLSSGFSPDFATVITMDRKASKQ |
| | | QTNIDVIYERVRDDYQLHWTSTNWKGTNTKDKWTDRSSERYKIDWEK |
| | | EEMTN |
| 14 | I5A, I7A, | ADSDANAKTGTTDIGSNTTVKTGDLVTYDKENGMLKKVFYSFIDDKNH |
| | H35L. | NKKLLVIRTKGTIAGQYCVYSCEGANKSGLAWPSAFKVQLQLPDNEVA |
| | R66C, | QISDYYPRNSIDTKEYMSTLTYGFNGNVTGDDTGKIGGLIGANVSIGHTL |
| | E70C | KYVQPDFKTILESPTDKKVGWKVIFNNMVNQNWGPYDRDSWNPVYGN |
| | | QLFMKTRNGSMKAADNFLDPNKASSLLSSGFSPDFATVITMDRKASKQ |
| | | QTNIDVIYERVRDDYQLHWTSTNWKGTNTKDKWTDRSSERYKIDWEK |
| | | EEMTN |
| 15 | K8A, | ADSDINIATGTTDIGSNTTVKTGDLVTYDKENGMLKKVFYSFIDDKNHNK |
| | H35L. | KLLVIRTKGTIAGQYCVYSEEGANKSGLAWPSAFKVQLQLPDNEVAQIS |
| | R66C, | DYYPRNSIDTKEYMSTLTYGFNGNVTGDDTGKIGGLIGANVSIGHTLKY |
| | | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 16 | K8A, | ADSDINIATGTTDIGSNTTVKTGDLVTYDKENGMLKKVFYSFIDDKNHNK |
| | H35L. | KLLVIRTKGTIAGQYRVYSCEGANKSGLAWPSAFKVQLQLPDNEVAQIS |
| | E70C | DYYPRNSIDTKEYMSTLTYGFNGNVTGDDTGKIGGLIGANVSIGHTLKY |
| | | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 17 | K8A, | ADSDINIATGTTDIGSNTTVKTGDLVTYDKENGMLKKVFYSFIDDKNHNK |
| | H35L, | KLLVIRTKGTIAGQYCVYSCEGANKSGLAWPSAFKVQLQLPDNEVAQIS |
| | R66C | DYYPRNSIDTKEYMSTLTYGFNGNVTGDDTGKIGGLIGANVSIGHTLKY |
| | E70C | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 18 | D45A | ADSDINIKTGTTDIGSNTTVKTGDLVTYDKENGMLKKVFYSFIDAKNHNK |
| | Y118F | KLLVIRTKGTIAGQYCVYSEEGANKSGLAWPSAFKVQLQLPDNEVAQIS |
| | H35L | DYYPRNSIDTKEYMSTLTFGFNGNVTGDDTGKIGGLIGANVSIGHTLKY |
| | R66C | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 19 | D45A | ADSDINIKTGTTDIGSNTTVKTGDLVTYDKENGMLKKVFYSFIDAKNHNK |
| | Y118F | KLLVIRTKGTIAGQYRVYSCEGANKSGLAWPSAFKVQLQLPDNEVAQIS |
| | H35L | DYYPRNSIDTKEYMSTLTFGFNGNVTGDDTGKIGGLIGANVSIGHTLKY |
| | E70C | VQPDFKTILESPTDKKVGWKVIFNNMVNQNWGPYDRDSWNPVYGNQL |
| | | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 20 | D45A | ADSDINIKTGTTDIGSNTTVKTGDLVTYDKENGMLKKVFYSFIDAKNHNK |
| | Y118F | KLLVIRTKGTIAGQYCVYSCEGANKSGLAWPSAFKVQLQLPDNEVAQIS |
| | H35L | DYYPRNSIDTKEYMSTLTFGFNGNVTGDDTGKIGGLIGANVSIGHTLKY |
| | R66C | VQPDFKTILESPTDKKVGWKVIFNNMVNQNWGPYDRDSWNPVYGNQL |
| | E70C | FMKTRNGSMKAADNFLDPNKASSLLSSGFSPDFATVITMDRKASKQQT |
| | | NIDVIYERVRDDYQLHWTSTNWKGTNTKDKWTDRSSERYKIDWEKEE |
| | | MTN |
| 21 | H35L, | ADSDANAATGTTDIGSNTTVKTGDLVTYDKENGMLKKVFYSFIDAKNH |
| | R66C, | NKKLLVIRTKGTIAGQYCVYSEEGANKSGLAWPSAFKVQLQLPDNEVA |
| | I5A, I7A, | QISDYYPRNSIDTKEYMSTLTFGFNGNVTGDDTGKIGGLIGANVSIGHTL |
| | D45A, | KYVQPDFKTILESPTDKKVGWKVIFNNMVNQNWGPYDRDSWNPVYGN |
| | Y118F | QLFMKTRNGSMKAADNFLDPNKASSLLSSGFSPDFATVITMDRKASKQ |
| | | QTNIDVIYERVRDDYQLHWTSTNWKGTNTKDKWTDRSSERYKIDWEK |
| | | EEMTN |
| 22 | H35L, | ADSDANAATGTTDIGSNTTVKTGDLVTYDKENGMLKKVFYSFIDAKNH |
| | E70C, | NKKLLVIRTKGTIAGQYRVYSCEGANKSGLAWPSAFKVQLQLPDNEVA |
| | I5A, I7A, | QISDYYPRNSIDTKEYMSTLTFGFNGNVTGDDTGKIGGLIGANVSIGHTL |
| | D45A, | KYVQPDFKTILESPTDKKVGWKVIFNNMVNQNWGPYDRDSWNPVYGN |
| | and | QLFMKTRNGSMKAADNFLDPNKASSLLSSGFSPDFATVITMDRKASKQ |
| | Y118F | QTNIDVIYERVRDDYQLHWTSTNWKGTNTKDKWTDRSSERYKIDWEK |
| | | EEMTN |
| 23 | H35L, | ADSDANAATGTTDIGSNTTVKTGDLVTYDKENGMLKKVFYSFIDAKNH |
| | R66C, | NKKLLVIRTKGTIAGQYCVYSCEGANKSGLAWPSAFKVQLQLPDNEVA |
| | E70C, | QISDYYPRNSIDTKEYMSTLTFGFNGNVTGDDTGKIGGLIGANVSIGHTL |
| | I5A, I7A, | KYVQPDFKTILESPTDKKVGWKVIFNNMVNQNWGPYDRDSWNPVYGN |
| | D45A, | QLFMKTRNGSMKAADNFLDPNKASSLLSSGFSPDFATVITMDRKASKQ |
| | Y118F | QTNIDVIYERVRDDYQLHWTSTNWKGTNTKDKWTDRSSERYKIDWEK |
| | | EEMTN |
| 24 | H35L, | ADSDINIKTGTTDIGSNTTVKTGDLVTYDKENGMLKKVFYSFIDDKNHNK |
| | R66C, | KLLVIRTKGTIAGQYCVYSEEGANKSGLAWPSAFKVQLQLPDNEVAQIS |
| | GPG | DYYPRNSIDTKEYMSTLTGPGPGKKVFYSFIDDKNHNKGPGPGSIGHT |
| | peptide | LKYVQPDFKTILESPTDKKVGWKVIFNNMVNQNWGPYDRDSWNPVYG |
| | | NQLFMKTRNGSMKAADNFLDPNKASSLLSSGFSPDFATVITMDRKASK |
| | | QQTNIDVIYERVRDDYQLHWTSTNWKGTNTKDKWTDRSSERYKIDWE |
| | | KEEMTN |
| 25 | H35L, | ADSDINIKTGTTDIGSNTTVKTGDLVTYDKENGMLKKVFYSFIDDKNHNK |
| | E70C, | KLLVIRTKGTIAGQYRVYSCEGANKSGLAWPSAFKVQLQLPDNEVAQIS |
| | GPG | DYYPRNSIDTKEYMSTLTGPGPGKKVFYSFIDDKNHNKGPGPGSIGHT |
| | peptide | LKYVQPDFKTILESPTDKKVGWKVIFNNMVNQNWGPYDRDSWNPVYG |
| | | NQLFMKTRNGSMKAADNFLDPNKASSLLSSGFSPDFATVITMDRKASK |
| | | QQTNIDVIYERVRDDYQLHWTSTNWKGTNTKDKWTDRSSERYKIDWE |
| | | KEEMTN |
| 26 | H35L, | ADSDINIKTGTTDIGSNTTVKTGDLVTYDKENGMLKKVFYSFIDDKNHNK |
| | R66C, | KLLVIRTKGTIAGQYCVYSCEGANKSGLAWPSAFKVQLQLPDNEVAQIS |
| | E70C, | DYYPRNSIDTKEYMSTLTGPGPGKKVFYSFIDDKNHNKGPGPGSIGHT |
| | GPG | LKYVQPDFKTILESPTDKKVGWKVIFNNMVNQNWGPYDRDSWNPVYG |
| | peptide | NQLFMKTRNGSMKAADNFLDPNKASSLLSSGFSPDFATVITMDRKASK |
| | | QQTNIDVIYERVRDDYQLHWTSTNWKGTNTKDKWTDRSSERYKIDWE |
| | | KEEMTN |
| 27 | I5A, I7A | ADSDANAKTGTTDIGSNTTVKTGDLVTYDKENGMLKKVFYSFIDDKNH |
| | H35L, | NKKLLVIRTKGTIAGQYCVYSEEGANKSGLAWPSAFKVQLQLPDNEVA |
| | R66C, | QISDYYPRNSIDTKEYMSTLTGPGPGKKVFYSFIDDKNHNKGPGPGSIG |
| | GPG | HTLKYVQPDFKTILESPTDKKVGWKVIFNNMVNQNWGPYDRDSWNPV |
| | peptide | YGNQLFMKTRNGSMKAADNFLDPNKASSLLSSGFSPDFATVITMDRKA |
| | | SKQQTNIDVIYERVRDDYQLHWTSTNWKGTNTKDKWTDRSSERYKID |
| | | WEKEEMTN |
| 28 | I5A, I7A | ADSDANAKTGTTDIGSNTTVKTGDLVTYDKENGMLKKVFYSFIDDKNH |
| | H35L, | NKKLLVIRTKGTIAGQYRVYSCEGANKSGLAWPSAFKVQLQLPDNEVA |
| | E70C, | QISDYYPRNSIDTKEYMSTLTGPGPGKKVFYSFIDDKNHNKGPGPGSIG |
| | GPG | HTLKYVQPDFKTILESPTDKKVGWKVIFNNMVNQNWGPYDRDSWNPV |
| | peptide | YGNQLFMKTRNGSMKAADNFLDPNKASSLLSSGFSPDFATVITMDRKA |
| | | SKQQTNIDVIYERVRDDYQLHWTSTNWKGTNTKDKWTDRSSERYKID |
| | | WEKEEMTN |
| 29 | I5A, I7A | ADSDANAKTGTTDIGSNTTVKTGDLVTYDKENGMLKKVFYSFIDDKNH |
| | H35L, | NKKLLVIRTKGTIAGQYCVYSCEGANKSGLAWPSAFKVQLQLPDNEVA |
| | R66C, | QISDYYPRNSIDTKEYMSTLTGPGPGKKVFYSFIDDKNHNKGPGPGSIG |
| | E70C, | HTLKYVQPDFKTILESPTDKKVGWKVIFNNMVNQNWGPYDRDSWNPV |
| | GPG | YGNQLFMKTRNGSMKAADNFLDPNKASSLLSSGFSPDFATVITMDRKA |
| | peptide | SKQQTNIDVIYERVRDDYQLHWTSTNWKGTNTKDKWTDRSSERYKID |
| | | WEKEEMTN |
| 30 | H35L, | ADSDINIKTGTTDIGSNTTVKTGDLVTYDKENGMLKKVFYSFIDAKNHNK |
| | D45A | KLLVIRTKGTIAGQYCVYSEEGANKSGLAWPSAFKVQLQLPDNEVAQIS |
| | R66C, | DYYPRNSIDTKEYMSTLTGPGPGKKVFYSFIDDKNHNKGPGPGSIGHT |
| | GPG | LKYVQPDFKTILESPTDKKVGWKVIFNNMVNQNWGPYDRDSWNPVYG |
| | peptide | NQLFMKTRNGSMKAADNFLDPNKASSLLSSGFSPDFATVITMDRKASK |
| | | QQTNIDVIYERVRDDYQLHWTSTNWKGTNTKDKWTDRSSERYKIDWE |
| | | KEEMTN |
| 31 | H35L, | ADSDINIKTGTTDIGSNTTVKTGDLVTYDKENGMLKKVFYSFIDAKNHNK |
| | D45A, | KLLVIRTKGTIAGQYRVYSCEGANKSGLAWPSAFKVQLQLPDNEVAQIS |
| | E70C, | DYYPRNSIDTKEYMSTLTGPGPGKKVFYSFIDDKNHNKGPGPGSIGHT |
| | GPG | LKYVQPDFKTILESPTDKKVGWKVIFNNMVNQNWGPYDRDSWNPVYG |
| | peptide | NQLFMKTRNGSMKAADNFLDPNKASSLLSSGFSPDFATVITMDRKASK |
| | | QQTNIDVIYERVRDDYQLHWTSTNWKGTNTKDKWTDRSSERYKIDWE |
| | | KEEMTN |
| 32 | H35L, | ADSDINIKTGTTDIGSNTTVKTGDLVTYDKENGMLKKVFYSFIDAKNHNK |
| | D45A, | KLLVIRTKGTIAGQYCVYSCEGANKSGLAWPSAFKVQLQLPDNEVAQIS |
| | R66C, | DYYPRNSIDTKEYMSTLTGPGPGKKVFYSFIDDKNHNKGPGPGSIGHT |
| | E70C, | LKYVQPDFKTILESPTDKKVGWKVIFNNMVNQNWGPYDRDSWNPVYG |
| | GPG | NQLFMKTRNGSMKAADNFLDPNKASSLLSSGFSPDFATVITMDRKASK |
| | peptide | QQTNIDVIYERVRDDYQLHWTSTNWKGTNTKDKWTDRSSERYKIDWE |
| | | KEEMTN |
| 33 | I5A, I7A | ADSDANAKTGTTDIGSNTTVKTGDLVTYDKENGMLKKVFYSFIDAKNH |
| | H35L, | NKKLLVIRTKGTIAGQYCVYSEEGANKSGLAWPSAFKVQLQLPDNEVA |
| | D45A | QISDYYPRNSIDTKEYMSTLTGPGPGKKVFYSFIDDKNHNKGPGPGSIG |
| | R66C, | HTLKYVQPDFKTILESPTDKKVGWKVIFNNMVNQNWGPYDRDSWNPV |
| | GPG | YGNQLFMKTRNGSMKAADNFLDPNKASSLLSSGFSPDFATVITMDRKA |
| | peptide | SKQQTNIDVIYERVRDDYQLHWTSTNWKGTNTKDKWTDRSSERYKID |
| | | WEKEEMTN |
| 34 | I5A | ADSDANAKTGTTDIGSNTTVKTGDLVTYDKENGMLKKVFYSFIDAKNH |
| | I7A | NKKLLVIRTKGTIAGQYRVYSCEGANKSGLAWPSAFKVQLQLPDNEVA |
| | H35L, | QISDYYPRNSIDTKEYMSTLTGPGPGKKVFYSFIDDKNHNKGPGPGSIG |
| | D45A, | HTLKYVQPDFKTILESPTDKKVGWKVIFNNMVNQNWGPYDRDSWNPV |
| | E70C, | YGNQLFMKTRNGSMKAADNFLDPNKASSLLSSGFSPDFATVITMDRKA |
| | GPG | SKQQTNIDVIYERVRDDYQLHWTSTNWKGTNTKDKWTDRSSERYKID |
| | peptide | WEKEEMTN |
| 35 | I5A, I7A | ADSDANAKTGTTDIGSNTTVKTGDLVTYDKENGMLKKVFYSFIDAKNH |
| | H35L, | NKKLLVIRTKGTIAGQYCVYSCEGANKSGLAWPSAFKVQLQLPDNEVA |
| | D45A, | QISDYYPRNSIDTKEYMSTLTGPGPGKKVFYSFIDDKNHNKGPGPGSIG |
| | R66C, | HTLKYVQPDFKTILESPTDKKVGWKVIFNNMVNQNWGPYDRDSWNPV |
| | E70C, | YGNQLFMKTRNGSMKAADNFLDPNKASSLLSSGFSPDFATVITMDRKA |
| | GPG | SKQQTNIDVIYERVRDDYQLHWTSTNWKGTNTKDKWTDRSSERYKID |
| | peptide | WEKEEMTN |
| 36 | | ADSDINIKTGTTDIGSNTTVKTGDLVTYDKENGMHKKVFYSFIDDKNHN |
| | | KGPGPG |
| 37 | | KKVFYSFIDDKNHNKGPGPGKKVFYSFIDDKNHNKGPGPGKKVFYSFID |
| | | DKNHNKGPGPGKKVFYSFIDDKNHNKGPGPGKKVFYSFIDDKNHNKG |
| | | PGPG |
| 38 | | KKVFYSFIDDKNHNKGPGPGTDKKVGWKVIFNNMVGPGPGKLLVIRTK |
| | | GTIAGQY |
| 39 | | GTATTTTATAGTTTTATCGATGCTAAAAATCACAATAAAA |
| 40 | | TTTTATTGTGATTTTTAGCATCGATAAAACTATAAAATAC |
| 41 | | GTATATGAGTACTTTAACTTTTGGATTCAACGGTAATGTTA |
| 42 | | TAACATTACCGTTGAATCCAAAAGTTAAAGTACTCATATAC |
| 43 | | CGGCGGCTCGAGATTTGTCATTTCTTCTTT |
| 44 | | CGGCGGTCTAGAAGGAGGATATATATGGCAGATTCTGATATAATATT |
| 45 | | CTTATGATAAAGAAAATGGCATGCTCAAAAAAGTATTTTATAGTTTTA |
| | | TCGATG |
| 46 | | CATCGATAAAACTATAAAATACTTTTTTGAGCATGCCATTTTCTTTATC |
| | | ATAAG |
| 47 | Mass | GTIAGQYCVYSCEGANK |
| | Spec | |
| | peptide | |

What is claimed is:

1-22. (canceled)

23. A modified α-hemolysin (Hla) polypeptide, comprising amino acid substitutions H35L, R66C and E70C relative to the amino acid sequence set forth in SEQ ID NO: 1.

24. The modified Hla polypeptide of claim 23, comprising an amino acid sequence at least 80% identical to the sequence set forth in SEQ ID NO: 1.

25. The modified Hla polypeptide of claim 24, comprising an amino acid sequence at least 95% identical to the sequence set forth in SEQ ID NO: 1.

26. A polynucleotide comprising a nucleic acid sequence encoding the modified Hla polypeptide of claim 23.

27. The polynucleotide sequence of claim 26, wherein the modified Hla polypeptide comprises an amino acid sequence at least 95% identical to the sequence set forth in SEQ ID NO: 1.

28. The polynucleotide sequence of claim 27, wherein the polynucleotide sequence is an isolated polynucleotide sequence, a plasmid, an expression vector, a cosmid, a viral vector, a virus, or a virus like particle (VLP).

29. A host cell comprising the polynucleotide sequence of claim 27.

30. The host cell of claim 29, wherein the host cell is any one of a Chinese hamster ovary (CHO) cell, a HEK 293 cell, a human cervical carcinoma cell (Hela), a canine kidney cell (MDCK), a human liver cell (HepG2), a baby hamster kidney cell (BHK), a monkey kidney cell (CV1), a Vero cell, a CEM cell, a 721.221 cell, a H9 cell, a Jurkat cell, a Raji cell, a W138 cell, a COS-7 cell, a 293 cell, a HepG2 cell, a 3T3 cell, and a RIN cell.

31. The Hla polypeptide of claim 23, comprising an amino acid sequence at least 95% identical to the sequence set forth in SEQ ID NO: 11.

32. The Hla polypeptide of claim 31, comprising the sequence set forth in SEQ ID NO: 11.

33. A composition comprising the modified Hla polypeptide of claim 23 and a pharmaceutically acceptable excipient.

34. The composition of claim 33, further comprising a pharmaceutically acceptable adjuvant.

35. The composition of claim 34, wherein the adjuvant is selected from alum, AddaSO3 (ASO3-like), MPLA and alum (ASO4-like), Fruend's, CpG-ODN1585, and any combination thereof.

36. The composition of claim 33, further comprising at least one additional active agent.

37. A composition comprising the Hla polypeptide of claim 32 and a pharmaceutically acceptable adjuvant.

38. A method of inducing an immune response against a bacterial pathogen in a subject in need thereof, comprising administering to the subject a composition comprising the modified Hla polypeptide, derivative, or fragment thereof of claim 23.

39. The method of claim 38, wherein the method enhances germinal center (GC) or T follicular helper (TFH) response or reduces the severity of skin and soft tissue infection, invasive Staphylococcus. aureus disease, sepsis, and carriage, as compared to administering an immunogenic composition comprising a modified Hla polypeptide with only a substitution H35L relative to the amino acid sequence set forth in SEQ ID NO: 1.

40. The method of claim 38, wherein the subject is a mammal.

41. The method of claim 40, wherein the mammal is a human.

42. The method of claim 38, wherein the subject is a child under 3 years of age, or under 2 years of age, or under 1 year of age, or less than 12 months, or 11 months, or 10 months, of 9 months, or 8 months, or 6 months, or 5 months, or 4 months, or 3 months, or 2 months, or 1 month, or less than 4 wks, or 3 wks, or 2 wks, or 1 wks in age.

43. The method of claim 38, wherein the subject is a pregnant woman.

44. The method of claim 38, wherein the bacterial pathogen is Staphylococcus aureus.

45. The method of claim 38, wherein the method further comprises administering the composition at least 1, at least 2, or at least 3 additional times.

46. The method of claim 38, further comprising administering at least one additional immunogenic antigen.

47. The method of claim 46, wherein the at least one additional immunogenic antigen is selected from Opp3a, DItD, HtsA, LtaS, IsdA, IsdB IsdC, SdrC, SdrD, SdrE, SdrF, SdrG, SdrH, SrtA, SpA, Sbi, FmtB, beta-hemolysin, fibronectin-binding protein A (FnbA), fibronectin-binding protein B (FnbB), coagulase, Fig, Map, Panton-Valentine leukocidin (Pvl), alpha-toxin and its variants, gamma toxin (hlg) and variants, Ica, immunodominant ABC transporter, Mg²⁺ transporter, Ni-ABC transporter, RAP, autolysin, laminin receptors, IsaA/PisA, IsaB/PisB, SPOIIIE, SsaA, EbpS, Sas A, SasF, SasH, EFB (FIB), SBI, Npase, EBP, bone sialo binding protein II, aureolysin precursor (AUR)/Sepp1, CNA, and fragments thereof such as M55, TSST-1, mecA, poly-N-acetylglucosamine (PNAG/dPNAG) exopolysaccharide, GehD, EbhA, EbhB, SSP-1, SSP-2, HBP, vitronectin binding protein, HarA, EsxA, EsxB, Enterotoxin A, Enterotoxin B, Enterotoxin C1, and novel autolysin.

48. The method of claim 38, wherein the administration is through an intravenous, intramuscular, sub-cutaneous, oral, or intraperitoneal route.

49. A method of reducing or preventing one or more symptoms associated with Staphylococcus aureus infection in a subject in need thereof, comprising administering to the subject the composition of claim 33.

50. The method of claim 49, wherein the one or more symptoms associated with Staphylococcus aureus infection is selected from skin and soft tissue infection, sepsis, abscess, and dermonecrosis.

51. A method of reducing or preventing a condition caused by Staphylococcus aureus infection in a subject in need thereof, comprising administering a composition comprising the modified Hla polypeptide, derivative, or fragment thereof of claim 23, wherein the composition is administered to (a) the mother of the subject while the subject is in utero; or (b) the subject at birth or shortly after birth.

52. The method of claim 51, the composition is administered to the subject (a) at birth; (b) within about 1, 2, 3, 4, 5, 10, 15, 20, 25, 30, 35, 40, 45, 50, 55 minutes after birth; (c) within about 1, 2, 3, 4, 5, 6, 7, 8, 9, 10, 11, 12, 13, 14, 15, 16, 17, 18, 19, 20, 21, 22, 23, 24 hours after birth; (d) within about 1, 2, 3, 4, 5, 6, 7 days after birth; or (e) about 1, 2, 3, 4, 5, 6, 7, or 8 weeks after birth.


SEQUENCE LISTING (47 sequences)

SEQ ID NO: 1 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 2 - DNA, 1485 bp, Staphylococcus aureus
  source 1..1485: mol_type = genomic DNA
atcgattacatttttaaatcaaacaaatcattagttttaaagaaattaatcaatagaattagctatgtcttttccttgtttcataaagaatttattcaactttgactaaccctcgaaattgaaatgcttcctttcaaattttaaataaaagttaaaaacatattcttaaaataatcactcatcatcactcagtaatttatcagttgctatattaaatatactaatataaacgattataaatatttgatatgtctcaactgcaaatattctaaattgacatattgattattgtttcctcaaatcgttcaaaaaaatagaaggatgatgaaaatgaaaacacgtatagtcagctcagtaacaacaacactattgctaggttccatattgatgaatcctgtcgctggtgccgcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaatgtaaattatttgtacatgtacaaataaatataatttataactttagccgataacttcagaaatgatgtttttcggctaatttttatacttaatttagtttaattaacttatgatatttttaatttaaactatgtacttgatttgctttcctgacttaaaaattagagctaaacatgacactatagaggtgtttt

SEQ ID NO: 3 - protein, 293 aa, synthetic construct
ADSDINIKTGTTDIGSNTTVKTGDLVTYDKENGMXKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 4 - protein, 15 aa, synthetic construct
KKVFYSFIDDKNHNK

SEQ ID NO: 5 - protein, 5 aa, synthetic construct
GPGPG

SEQ ID NO: 6 - protein, 293 aa, synthetic construct
ADSDINIKTGTTDIGSNTTVKTGDLVTYDKENGMXKKVFYSFIDDKNHNKKLLVIRTKGTIAGQYCVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 7 - protein, 293 aa, synthetic construct
ADSDINIKTGTTDIGSNTTVKTGDLVTYDKENGMLKKVFYSFIDDKNHNKKLLVIRTKGTIAGQYCVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 8 - protein, 293 aa, synthetic construct
ADSDINIKTGTTDIGSNTTVKTGDLVTYDKENGMXKKVFYSFIDDKNHNKKLLVIRTKGTIAGQYRVYSCEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 9 - protein, 293 aa, synthetic construct
ADSDINIKTGTTDIGSNTTVKTGDLVTYDKENGMLKKVFYSFIDDKNHNKKLLVIRTKGTIAGQYRVYSCEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 10 - protein, 293 aa, synthetic construct
ADSDINIKTGTTDIGSNTTVKTGDLVTYDKENGMXKKVFYSFIDDKNHNKKLLVIRTKGTIAGQYCVYSCEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 11 - protein, 293 aa, synthetic construct
ADSDINIKTGTTDIGSNTTVKTGDLVTYDKENGMLKKVFYSFIDDKNHNKKLLVIRTKGTIAGQYCVYSCEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 12 - protein, 293 aa, synthetic construct
ADSDANAKTGTTDIGSNTTVKTGDLVTYDKENGMLKKVFYSFIDDKNHNKKLLVIRTKGTIAGQYCVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 13 - protein, 293 aa, synthetic construct
ADSDANAKTGTTDIGSNTTVKTGDLVTYDKENGMLKKVFYSFIDDKNHNKKLLVIRTKGTIAGQYRVYSCEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 14 - protein, 293 aa, synthetic construct
ADSDANAKTGTTDIGSNTTVKTGDLVTYDKENGMLKKVFYSFIDDKNHNKKLLVIRTKGTIAGQYCVYSCEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 15 - protein, 293 aa, synthetic construct
ADSDINIATGTTDIGSNTTVKTGDLVTYDKENGMLKKVFYSFIDDKNHNKKLLVIRTKGTIAGQYCVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 16 - protein, 293 aa, synthetic construct
ADSDINIATGTTDIGSNTTVKTGDLVTYDKENGMLKKVFYSFIDDKNHNKKLLVIRTKGTIAGQYRVYSCEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 17 - protein, 293 aa, synthetic construct
ADSDINIATGTTDIGSNTTVKTGDLVTYDKENGMLKKVFYSFIDDKNHNKKLLVIRTKGTIAGQYCVYSCEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 18 - protein, 293 aa, synthetic construct
ADSDINIKTGTTDIGSNTTVKTGDLVTYDKENGMLKKVFYSFIDAKNHNKKLLVIRTKGTIAGQYCVYSEEGANKSGLAWPSAFKVQLQLPDNEVAQISDYYPRNSIDTKEYMSTLTFGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 19 - protein, 293 aa, synthetic construct
ADSDINIKTGTTDIGSNTTVKTGDLVTYDKENGMLKKVFYSFIDAKNHNKKLLVIRTKGTIAGQYRVYSCEGANKSGLAWPSAFKVQLQLPDNEVAQISDYYPRNSIDTKEYMSTLTFGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 20 - protein, 293 aa, synthetic construct
ADSDINIKTGTTDIGSNTTVKTGDLVTYDKENGMLKKVFYSFIDAKNHNKKLLVIRTKGTIAGQYCVYSCEGANKSGLAWPSAFKVQLQLPDNEVAQISDYYPRNSIDTKEYMSTLTFGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 21 - protein, 293 aa, synthetic construct
ADSDANAATGTTDIGSNTTVKTGDLVTYDKENGMLKKVFYSFIDAKNHNKKLLVIRTKGTIAGQYCVYSEEGANKSGLAWPSAFKVQLQLPDNEVAQISDYYPRNSIDTKEYMSTLTFGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 22 - protein, 293 aa, synthetic construct
ADSDANAATGTTDIGSNTTVKTGDLVTYDKENGMLKKVFYSFIDAKNHNKKLLVIRTKGTIAGQYRVYSCEGANKSGLAWPSAFKVQLQLPDNEVAQISDYYPRNSIDTKEYMSTLTFGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 23 - protein, 293 aa, synthetic construct
ADSDANAATGTTDIGSNTTVKTGDLVTYDKENGMLKKVFYSFIDAKNHNKKLLVIRTKGTIAGQYCVYSCEGANKSGLAWPSAFKVQLQLPDNEVAQISDYYPRNSIDTKEYMSTLTFGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 24 - protein, 295 aa, synthetic construct
ADSDINIKTGTTDIGSNTTVKTGDLVTYDKENGMLKKVFYSFIDDKNHNKKLLVIRTKGTIAGQYCVYSEEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 25 - protein, 295 aa, synthetic construct
ADSDINIKTGTTDIGSNTTVKTGDLVTYDKENGMLKKVFYSFIDDKNHNKKLLVIRTKGTIAGQYRVYSCEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 26 - protein, 295 aa, synthetic construct
ADSDINIKTGTTDIGSNTTVKTGDLVTYDKENGMLKKVFYSFIDDKNHNKKLLVIRTKGTIAGQYCVYSCEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 27 - protein, 295 aa, synthetic construct
ADSDANAKTGTTDIGSNTTVKTGDLVTYDKENGMLKKVFYSFIDDKNHNKKLLVIRTKGTIAGQYCVYSEEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 28 - protein, 295 aa, synthetic construct
ADSDANAKTGTTDIGSNTTVKTGDLVTYDKENGMLKKVFYSFIDDKNHNKKLLVIRTKGTIAGQYRVYSCEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 29 - protein, 295 aa, synthetic construct
ADSDANAKTGTTDIGSNTTVKTGDLVTYDKENGMLKKVFYSFIDDKNHNKKLLVIRTKGTIAGQYCVYSCEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 30 - protein, 295 aa, synthetic construct
ADSDINIKTGTTDIGSNTTVKTGDLVTYDKENGMLKKVFYSFIDAKNHNKKLLVIRTKGTIAGQYCVYSEEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 31 - protein, 295 aa, synthetic construct
ADSDINIKTGTTDIGSNTTVKTGDLVTYDKENGMLKKVFYSFIDAKNHNKKLLVIRTKGTIAGQYRVYSCEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 32 - protein, 295 aa, synthetic construct
ADSDINIKTGTTDIGSNTTVKTGDLVTYDKENGMLKKVFYSFIDAKNHNKKLLVIRTKGTIAGQYCVYSCEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 33 - protein, 295 aa, synthetic construct
ADSDANAKTGTTDIGSNTTVKTGDLVTYDKENGMLKKVFYSFIDAKNHNKKLLVIRTKGTIAGQYCVYSEEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 34 - protein, 295 aa, synthetic construct
ADSDANAKTGTTDIGSNTTVKTGDLVTYDKENGMLKKVFYSFIDAKNHNKKLLVIRTKGTIAGQYRVYSCEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 35 - protein, 295 aa, synthetic construct
ADSDANAKTGTTDIGSNTTVKTGDLVTYDKENGMLKKVFYSFIDAKNHNKKLLVIRTKGTIAGQYCVYSCEGANKSGLAWPSAFKVQLQLPDNEVAQISDYYPRNSIDTKEYMSTLTGPGPGKKVFYSFIDDKNHNKGPGPGSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 36 - protein, 55 aa, synthetic construct
ADSDINIKTGTTDIGSNTTVKTGDLVTYDKENGMHKKVFYSFIDDKNHNKGPGPG

SEQ ID NO: 37 - protein, 100 aa, synthetic construct
KKVFYSFIDDKNHNKGPGPGKKVFYSFIDDKNHNKGPGPGKKVFYSFIDDKNHNKGPGPGKKVFYSFIDDKNHNKGPGPGKKVFYSFIDDKNHNKGPGPG

SEQ ID NO: 38 - protein, 55 aa, synthetic construct
KKVFYSFIDDKNHNKGPGPGTDKKVGWKVIFNNMVGPGPGKLLVIRTKGTIAGQY

SEQ ID NO: 39 - DNA, 40 bp, synthetic construct
  source 1..40: mol_type = other DNA
gtattttatagttttatcgatgctaaaaatcacaataaaa

SEQ ID NO: 40 - DNA, 40 bp, synthetic construct
  source 1..40: mol_type = other DNA
ttttattgtgatttttagcatcgataaaactataaaatac

SEQ ID NO: 41 - DNA, 41 bp, synthetic construct
  source 1..41: mol_type = other DNA
gtatatgagtactttaacttttggattcaacggtaatgtta

SEQ ID NO: 42 - DNA, 41 bp, synthetic construct
  source 1..41: mol_type = other DNA
taacattaccgttgaatccaaaagttaaagtactcatatac

SEQ ID NO: 43 - DNA, 30 bp, synthetic construct
  source 1..30: mol_type = other DNA
cggcggctcgagatttgtcatttcttcttt

SEQ ID NO: 44 - DNA, 47 bp, synthetic construct
  source 1..47: mol_type = other DNA
cggcggtctagaaggaggatatatatggcagattctgatataatatt

SEQ ID NO: 45 - DNA, 54 bp, synthetic construct
  source 1..54: mol_type = other DNA
cttatgataaagaaaatggcatgctcaaaaaagtattttatagttttatcgatg

SEQ ID NO: 46 - DNA, 54 bp, synthetic construct
  source 1..54: mol_type = other DNA
catcgataaaactataaaatacttttttgagcatgccattttctttatcataag

SEQ ID NO: 47 - protein, 17 aa, synthetic construct
GTIAGQYCVYSCEGANK